(19) United States
(12) Patent Application Publication
(10) Pub. No.: US 2007/0271634 A1
(43) Pub. Date: Nov. 22, 2007

(54) Preventing stress-induced apoptosis in plants

(76) Inventor: Martin B. Dickman, Lincoln, NE (US)

Correspondence Address: BIOTECHNOLOGY LAW GROUP;C/O PORTFOLIOIP, PO BOX 52050, MINNEAPOLIS, MN, 55402, US

(21) Appl. No.: 11/436,695

(22) Filed: May 18, 2006

(51) Int. Cl. A01H 5/00; C12Q 1/68; C12N 15/82; C12N 5/04

(52) U.S. Cl. 800/288; 435/419; 435/6; 435/254.2

(57) ABSTRACT

The invention relates to methods of identifying nucleic acids that modulate apoptosis in plants, particularly apoptosis induced by an abiotic or biotic stress, as well as to transgenic plants and plant cells modified to include such nucleic acids.

### CROSS-REFERENCE TO RELATED APPLICATIONS

This application claims the benefit of and priority to each of the following commonly owned patent applications: U.S. provisional patent application Ser. No. 60/106,321, filed 30 Oct. 1998; U.S. provisional patent application Ser. No. 60/138,303, filed 9 Jun. 1999; U.S. non-provisional patent application Ser. No. 09/430,231, filed 29 Oct. 29, 1999; and U.S. non-provisional patent application Ser. No. 11/223,254, filed 9 Sep. 2005, each of which is hereby incorporated by reference in its entirety for all purposes.

This invention was made at least in part with government support. As a result, the U.S. government may have certain rights in this invention.

### TECHNICAL FIELD

The present invention relates generally to modulating apoptosis in plants, and more particularly, to the novel transfer of genes encoding apoptotic pathway proteins from non-plant species into plants, thereby generating plants with increased resistance to various biotic and abiotic insults as well as providing other related advantages.

### BACKGROUND OF THE INVENTION

With recent advances in understanding the complex signaling pathways which induce programmed cell death in animal cells, research has intensified in identifying similar pathways in evolutionarily distant organisms, such as plants. In plants, programmed cell death-regimes are recognized to occur at specific points during development, and may be triggered in response to pathogens (see, Woodson et al., Plant Physiol. 99:526-532, 1992; O'Neill et al., The Plant Cell 5:419-432, 1993; Chasan, The Plant Cell 6:917-919, 1994; Smart, New Phytol. 126:419-448, 1994; Keen, Ann. Rev. Genet. 24:447-463, 1990; Lamb, Cell 76:419-422, 1994; Mittler et al., Cell 7:29-42, 1995). Among the disease-related circumstances in which cell death has been suggested to play a determinative role is the hypersensitive resistance-associated (HR) cell death that is characteristic of several incompatible plant-microbe interactions. While morphological evidence demonstrating the hallmarks of apoptosis, including DNA ladders and apoptotic bodies (Wyllie, Curr. Opin. Gen. Dev. 5:97-104, 1995) have now been reported in plant development, disease associated death, and in the hypersensitive reaction (Wang et al., The Plant Cell 8:375-391, 1996; Ryerson and Heath, The Plant Cell 8:393-402, 1996), very little is known about the genes and corresponding proteins that control plant cell death and whether the animal genes (vertebrate and invertebrate) that are known to control apoptosis in animal cells have homologues in plants.

Within animal tissues, homeostasis is maintained by the process of apoptosis—that is, the normal physiological process of programmed cell death. Changes to the apoptotic pathway that prevent or delay normal cell turnover can be just as important in the pathogenesis of diseases as are abnormalities in the regulation of the cell cycle. Like cell division, which is controlled through complex interactions between cell cycle regulatory proteins, apoptosis is similarly regulated under normal circumstances by the interaction of gene products that either prevent or induce cell death.

Since apoptosis functions in maintaining tissue homeostasis in a range of physiological processes such as embryonic development, immune cell regulation and normal cellular turnover, the dysfunction or loss of regulated apoptosis can lead to a variety of pathological disease states. For example, the loss of apoptosis can lead to the pathological accumulation of self-reactive lymphocytes that occurs with many autoimmune diseases. Inappropriate loss or inhibition of apoptosis can also lead to the accumulation of virally infected cells and of hyperproliferative cells such as neoplastic or tumor cells. Similarly, the inappropriate activation of apoptosis can also contribute to a variety of pathological disease states including, for example, acquired immunodeficiency syndrome (AIDS), neurodegenerative diseases and ischemic injury. Treatments which are specifically designed to modulate the apoptotic pathways in these and other pathological conditions can alter the natural progression of many of these diseases. Accordingly, given the recognized importance of apoptosis in animals and its reported importance in development and pathogen resistance in plants (Mittler, in: When Cells Die, Lockshin et al. (eds.), pp. 147-174, 1998, understanding apoptotic pathways in such organisms as plants could be valuable.

Although apoptosis is mediated by diverse signals and complex interactions of cellular gene products, the results of these interactions ultimately feed into a cell death pathway that is evolutionarily conserved between humans and invertebrates. The pathway, itself, includes a cascade of proteolytic zymogen activation events analogous to that of the blood coagulation cascade. Interestingly, efforts to identify an analogous pathway in plants has yet to come to fruition and the relationship between mammalian and invertebrate apoptosis with that of programmed cell death pathways in plants remains ambiguous, at best. However, the hypersensitive response as well as plant senescence demonstrate characteristics consistent with a programmed cell death pathway (Drake et al., Plant Mol. Biol. 30:755-767, 1996). Accordingly, understanding the programmed cell death pathway of plants may lead to methods of regulating the same and accordingly, to transgenic plants harboring cell death modulators that have unique phenotypic characteristics, such as resistance to various biotic and abiotic insults, as well as increased shelf-life of cut plants, fruits, and vegetables.

Therefore, there exists a need in the art for methods of identifying cell death pathway components in plants as well as for methods of modulating programmed cell death in plants. Further, there exists a need in the art to produce biotic and abiotic insult resistant plants to improve crop yield and vitality. There also exists a need in the art to produce plants and plant products with decreased senescence. The present invention fulfills this need through the trans-species transfer of genes encoding apoptotic pathway proteins, allowing for modulation of plant programmed cell death pathways for the prevention of plant cell pathology and senescence, as well as providing methods and compositions for assaying compounds for apoptotic inhibitory and enhancing ability, while further providing other related advantages.

### SUMMARY OF THE INVENTION

Briefly stated, the present invention provides methods for modulating apoptosis in plants through the use of nucleotide sequences encoding biologically functional apoptotic pathway proteins.

In one aspect, the present invention provides a transgenic plant, comprising plant cells containing at least one heterologous nucleotide sequence capable of encoding a biologically functional apoptotic pathway protein or a functional variant thereof. In certain embodiments, the transgenic plant has a nucleotide sequence that encodes an anti-apoptotic protein. In various embodiments, the nucleotide sequence may encode Ced-9, Bcl-2, IAP, E1B 19K, and functional variants thereof. In yet other embodiments, the expression of the apoptotic pathway protein is controlled by tissue specific, inducible, or constitutive promoters. In certain other embodiments, the plant is pathogen or senescence resistant. As discussed in greater detail below, further aspects of the present invention provide various biotic and abiotic resistant plants.

The present invention also provides plant cells and seeds, containing a heterologous nucleotide sequence capable of encoding a biologically functional apoptotic pathway protein or a functional variant thereof. In other various embodiments, plant cells are provided that have the same characteristics as noted above for a transgenic plant.

As noted above, the present invention provides transgenic biotic insult resistant plants. The plants contain a heterologous nucleic acid sequence encoding a biologically functional apoptotic pathway protein, preferably an anti-apoptotic protein. In certain embodiments, the anti-apoptotic protein is selected from the group consisting of Ced-9, Bcl-2, IAP, E1B 19K, and homologs thereof. In the various embodiments, the biotic insult is the result of a pathogen.

Within a related aspect, transgenic abiotic insult resistant plants are provided.

The plants contain a heterologous nucleic acid sequence encoding a biologically functional apoptotic pathway protein, preferably an anti-apoptotic protein. In certain embodiments, the anti-apoptotic protein is selected from the group consisting of Ced-9, Bcl-2, IAP, E1B 19K, and functional variants thereof. In addition, the present invention provides methods of generating such transgenic biotic or abiotic resistant plants, comprising transforming a plant cell with a vector comprising at least one heterologous nucleic acid sequence encoding a biologically functional apoptotic pathway protein, the nucleic acid sequence operably associated with a promoter, producing a plant from the transformed plant cells, and selecting a transformed plant having biotic or abiotic resistance.

In still other aspects of the present invention, methods are provided for modulating apoptosis in a plant, comprising transforming a plant cell with a vector comprising a nucleic acid sequence encoding a biologically functional apoptotic pathway protein not normally produced by the plant cell, the nucleic acid sequence operably associated with an inducible promoter, culturing the transformed plant cell under conditions suitable for the formation of a plant, and growing the plant under conditions and for a time sufficient to induce transcription of the nucleic acid sequence.

Methods of identifying plant genes having apoptotic pathway activity are also provided, comprising transforming animal cell(s) with a plant cDNA library, wherein each member of the cDNA library is operably associated with a promoter, contacting the transformed cell(s) with an apoptotic inducer, and detecting apoptotic activity in the cells, and comparing this activity to a control cell line, wherein an increase or decrease in activity indicates the presence of an apoptotic pathway protein in the plant cDNA library.

In related aspects, the present invention provides, methods of identifying an apoptotic gene that functions in plants, comprising transforming one or more plant cell(s) with at least one heterologous nucleic acid molecule, wherein the nucleic acid molecule is operably associated with a promoter, contacting the transformed cell(s) with an apoptotic inducer, and detecting apoptotic activity in the cells, and comparing this activity to a control cell line, wherein an increase or decrease in activity indicates the presence of an apoptotic pathway gene that functions in plants. In certain embodiments, the heterologous nucleic acid molecule comprises a heterologous cDNA library, wherein each member of the cDNA library is operably associated with a promoter.

The transgenic plants, plant cells, and methods of the present invention as described above have general applicability to plant programmed cell death. Accordingly, methods of identifying cell death pathway components in plants as well as for methods of modulating programmed cell death in plants are provided. As noted above, the present invention also provides biotic and abiotic insult resistant plants to improve crop yield and vitality. Moreover, plants and plant products with decreased senescence are provided leading to longer shelf-lives of cut fruits and vegetables as well as cut flowers, etc. In addition, the present invention has applicability to methods and compositions for assaying compounds for apoptotic inhibitory and enhancing ability, while further providing other related advantages.

These and other aspects of the present invention will become evident upon reference to the following detailed description and attached drawings. In addition, various references are set forth below which describe in more detail certain procedures or compositions (e.g., plasmids, etc.), and are therefore incorporated by reference in their entireties.

### BRIEF DESCRIPTION OF THE DRAWINGS

FIGS. 1A and 1B are schematic representations of plasmids harboring an IAP encoding nucleic acid molecule.

FIGS. 2A and 2B are schematic representations of plasmids harboring a Bcl-2 encoding nucleic acid molecule.

FIGS. 3A and 3B are schematic representations of plasmids harboring a Ced-9 encoding nucleic acid molecule.

FIGS. 4A-4C are schematic representations of plasmids harboring a E1B-19K encoding nucleic acid molecule.

FIG. 5 is a scanned image representing Northern Blot analysis of transgene expression in transformed tobacco plants. Lanes 1-3 represent expression of IAP in separate tobacco plants, lane 4 represents expression in a tobacco plant transformed with a control vector, lanes 5-6 represent expression of Ced-9 in separate tobacco plants.

FIG. 6 is a photograph of leaf morphology following inoculation of three transgenic tobacco lines (A, C, D) and a control line (B) with Sclerotinia sclerotiorum.

FIG. 7 is a photograph of leaf morphology following inoculation of wild-type controls (A, B, C) and two independent IAP expressing transgenic tobacco lines (D and E) with tobacco mosaic virus (TMV).

FIG. 8 is a scanned image representing Northern Blot analysis of primary tobacco transformants (Glurk cultivar) harboring Bcl-2. Lanes 2, 3, 5, and 7 demonstrate Bcl-2 expression, while the control lane represents transformation with a vector (G115) with no Bcl-2 insert.

FIG. 9 is a scanned image representing Northern Blot analysis of primary tobacco transformants harboring Ced-9. Lanes 4 and 8 demonstrate expression.

FIGS. 10A and 10B are photographs of leaf morphology following inoculation of Bcl-2 harboring tobacco with TMV. The center leaf is wild-type while the remaining leaves are derived from separate Bcl-2 harboring tobacco plants.

FIGS. 11A and 11B are scanned images of Western Blot analysis of TMV expression in N gene containing (A) and N gene absent (B) tobacco lines harboring Bcl-2 or Ced-9 genes. B is Bcl-2, C is Ced-9, T is TMV, and A represents leaf samples taken from sections adjacent to inoculation site.

FIG. 12 is a photograph of leaf morphology of Bcl-2 and Ced-9 harboring tobacco plants following inoculation with tomato spotted wilt virus (TSWV). Upper and lower right leaves are controls, while the upper left leaf is a Glurk cultivar harboring Ced-9 and the lower left leaf is a Turkish cultivar harboring Ced-9.

FIG. 13 is a scanned image of Northern Blot analysis of Ced-9 expression of a selfed transgenic tobacco plant. Lanes 1-4 are individual progeny plants.

FIGS. 14A to 14C are photographs of leaf morphology of selfed transgenic tobacco plants. 14A depicts Bcl-2 harboring plant resistance to TMV, center leaf being a positive control, 14B depicts Ced-9 harboring plant resistance to TMV, and 14C depicts Ced-9 harboring plant resistance to TSWV.

FIG. 15 is a photograph of leaf morphology of S. sclerotiorum inoculated leaves. Lower leaves are wild-type controls, upper leaves are Ced-9 harboring.

FIGS. 16A and 16B are photographs of leaf morphology of selfed transgenic Bcl-2 harboring (A) and Ced-9 harboring (B) plants following inoculation with Sclerotinia.

FIGS. 17A-17C are photographs of leaf morphology of Bcl-2 harboring tobacco lines (A) and Ced-9 harboring tobacco lines (B and C) inoculated with Botrytis cinerea.

FIG. 18 is a scanned image of Northern Blot analysis of Ced-9 expression in Sclerotinia resistant plants (lanes 5-7) and non-resistant plants (lanes 1-4).

FIG. 19 is a photograph of leaf morphology of Bcl-2, IAP, Ced-9, E1B 19K and control (vector only); left to right respectively, following inoculation with Cercospora nicotianae.

FIGS. 20A and 20B are photographs of leaf morphology of Bcl-xL harboring tobacco plants. In 20A, the top leaves harbor a G138A mutation in Bcl-xL, while the bottom leaves harbor wild-type Bcl-xL. In 20B, the lefthand leaves harbor a G138A mutation in Bcl-xL, while the righthand leaves harbor wild-type Bcl-xL

### DETAILED DESCRIPTION OF THE INVENTION

Prior to setting forth the invention, it may be helpful to an understanding thereof to set forth definitions of certain terms that will be used hereinafter.

A “necrotroph”, as used herein, refers to a pathogen that kills a cell to complete its lifecycle and gains nutrition by use of dead cell material (e.g., many fungi).

A “biotic insult”, as used herein, refers to plant challenge caused by viable or biologic agents (biotic agents). Accordingly, biotic agents which cause a biotic insult include, for example, insects, fungi, bacteria, viruses, nematodes, viroids, mycloplasmas, etc.

An “abiotic insult”, as used herein, refers to plant challenge by a non-viable or non-living agent (abiotic agent). Accordingly, abiotic agents which cause an abiotic insult include, for example, environmental factors such as low moisture (drought), high moisture (flooding), nutrient deficiency, radiation levels, air pollution (ozone, acid rain, sulfur dioxide, etc.), temperature (hot and cold extremes), and soil toxicity, as well as herbicide damage, pesticide damage, or other agricultural practices (e.g., over-fertilization, improper use of chemical sprays, etc.).

An “apoptotic pathway protein”, as used herein, refers to any one of the proteins that are involved in the programmed cell death pathway as has been elucidated in a number of organisms, including human, mouse, C. elegans, Drosophila Melanogaster, and baculovirus proteins, excluding the p35 gene of baculovirus. In addition, the phrases “apoptotic pathway protein encoding gene” or “apoptotic pathway gene” are used interchangeably herein. A variety of apoptotic pathway proteins are known and are useful within the context of the present invention. Exemplary molecules include caspase molecules (currently 14 of which have been identified), bcl-2 family members (e.g., bcl-2, bcl-x_{L}, bcl-x_{S}, bcl-W, McL-1, A1, NR-13, Ced-9, E1B 19K, BHRF1, KSHV ORF 16, LMW5-HL, KS-bcl-2, etc.), Bax, Bak, Bok, Bad, Bik, Bid, Blk, Hrk, BNIP3, Bim_{L}, EGL-1, Inhibitors of Apoptosis (e.g., IAP-baculovirus, DIAP 1 & 2-Drosophila, c-IAP 1 & 2-human, X-IAP-human, NIAP-human, survivin-human, etc.), and the like. The sequences for these and other genes are available from the Genbank Database and are described in numerous publications including When Cells Die, edited by Lockshin et al., Wiley-Liss, New York, 1998.

As used herein, a “rev-caspase” refers to a cysteine protease that specifically cleaves proteins after Asp residues and is expressed as a zymogen, in which a small subunit is N-terminal to a large subunit. (see, e.g., PCT WO 99/00632)

Within the context of this invention, it should be understood that apoptotic pathway proteins include wild-type protein sequences, as well as other variants (including alleles) of the native protein sequence. Briefly, such variants may result from natural polymorphisms or may be synthesized by recombinant methodology, and differ from wild-type protein by one or more amino acid substitutions, insertions, deletions, or the like. Typically, when engineered, amino acid substitutions will be conservative, i.e., substitution of amino acids within groups of polar, non-polar, aromatic, charged, etc. amino acids. In the region of homology to the native sequence, variants should preferably have at least 90% amino acid sequence identity, and within certain embodiments, greater than 92%, 95%, or 97% identity. Such amino acid sequence and nucleic acid identity may be determined by standard methodologies, including use of the National Center for Biotechnology Information BLAST search methodology available at www.ncbi.nlm.nih.gov. The identity methodologies preferred are non-gapped BLAST algorithms. However, the identity algorithms described in U.S. Pat. No. 5,691,179 and Altschul et al., Nucleic Acids Res. 25:3389-3402, 1997 are also useful and are incorporated herein by reference. Accordingly, if Gapped BLAST 2.0 is utilized, then it is utilized with default settings. Further, a nucleotide variant will typically be sufficiently similar in sequence to hybridize to the reference sequence under stringent hybridization conditions (for nucleic acid molecules over about 500 bp, stringent conditions include a solution comprising about 1 M Na⁺ at 25° to 30° C. below the Tm; e.g., 5×SSPE, 0.5% SDS, at 65° C.; see, Ausubel, et al., Current Protocols in Molecular Biology, Greene Publishing, 1995; Sambrook et al., Molecular Cloning: A Laboratory Manual, Cold Spring Harbor Press, 1989). Some variants may not hybridize to the reference sequence because of codon degeneracy, such as degeneracies introduced for codon optimization in a particular host, in which case amino acid identity may be used to assess similarity of the variant to the reference protein.

An “isolated nucleic acid molecule” refers to a polynucleotide molecule in the form of a separate fragment or as a component of a larger nucleic acid construct, that has been separated from its source cell (including the chromosome it normally resides in) at least once in a substantially pure form. Nucleic acid molecules may be comprised of a wide variety of nucleotides, including DNA, RNA, nucleotide analogues, or some combination of these.

The term “in vitro” refers to systems comprising less than a whole cell.

The term “ex vivo” refers to cell culture systems, which include, for example, primary and secondary cell culture, whole organ culture, and similar systems.

The term “in vivo” refers to whole organisms.

“Genetic modification”, as used herein, refers to the introduction of one or more heterologous nucleic acid sequences into one or more plant cells, which can generate whole, sexually competent, viable plants. The terms “transgenic” and “genetically modified” are used interchangeably herein to refer to a plant which has been generated through the aforementioned process. Transgenic plants of the present invention are capable of self-pollinating or cross-pollinating with other plants of the same species so that the foreign gene, carried in the germ line, can be inserted into or bred into agriculturally useful plant varieties.

The term “plant cell”, as used herein, refers to protoplasts, gamete producing cells, and cells which are capable of regenerating into whole plants. Accordingly, a seed comprising multiple plant cells capable of regenerating into a whole plant is included in the definition of “plant cell”.

As used herein, “plant tissue” includes differentiated and undifferentiated tissues of a plant, including but not limited to roots, stems, shoots, leaves, pollen, seeds, tumor tissue and various forms of cells and culture such as single cells, protoplast, embryos, and callus tissue.

As used herein, the term “plant” refers to either a whole plant, a plant part, or a group of plant cells, such as plant tissue, for example. Plantlets are also included within the meaning of “plant”. Suitable plants for use in the invention include any plants amenable to transformation techniques, including both monocotyledonous and dicotyledonous plants.

Examples of monocotyledonous plants include, but are not limited to, asparagus, field and sweet corn, barley, wheat, rice, sorghum, onion, pearl millet, rye and oat, and ornamentals. Examples of dicotyledonous plants include, but are not limited to, tomato, potato, arabidopsis, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers and various ornamentals. The tobacco plant is used herein as an exemplary plant.

The term “heterologous nucleic acid sequence”, as used herein, refers to at least one structural gene, the gene generally operably associated with a regulatory sequence such as a promoter. The nucleic acid sequence originates in a foreign species, or in the same species if substantially modified from its original form. For example, the term “heterologous nucleic acid sequence” includes a nucleic acid originating in the same species, where such sequence is operably associated with a promoter that differs from the natural or wild-type promoter.

A “structural gene” is a DNA sequence that is transcribed into messenger RNA (mRNA) which is then translated into a sequence of amino acids characteristic of a specific polypeptide. The term “expression” refers to biosynthesis of a gene product.

The term “operably associated” refers to functional linkage between a promoter sequence and the structural gene regulated by the promoter nucleic acid sequence. The operably associated promoter controls the expression of the polypeptide encoded by the structural gene.

A “promoter”, as used herein, refers to a DNA sequence that directs the transcription of a structural gene. Typically a promoter is located in the 5′ region of a gene, proximal to the transcriptional start site of a structural gene.

A “cloning vector”, as used herein, refers to a DNA molecule such as a plasmid, cosmid, or bacteriophage that has the capability of replicating autonomously in a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector.

An “expression vector”, as used herein, refers to a DNA molecule, comprising a gene that is expressed in a host cell. Typically gene expression is placed under the control of certain regulatory elements including promoters, tissue specific regulatory elements, and enhancers. Such a gene is, as noted above, “operably associated” with the regulatory elements.

As used herein, a “pathogen”, refers to any agent that causes a disease or disease state in a plant, including, but not limited to viruses, fungi, bacterium, nematodes, and other related microorganisms.

As used herein, “modulate”, refers to the ability to alter or change from basal level.

As used herein, “inhibit”, refers to the ability to block, delay, or reduce the severity of an activity or result in a statistically significant fashion.

A. Apoptotic Pathway Genes and Gene Products

As noted above, the instant invention provides transgenic plants and plant tissues containing apoptotic pathway protein encoding genes, thereby allowing expression of the genes. A variety of genes encoding apoptotic pathway proteins are known and useful within the context of the present invention. Exemplary molecules include caspase molecules (currently 14 of which have been identified), Apaf-1 (Genbank NM 001160 and AF013263), bcl-2 family members (e.g., bcl-2, bcl-x_{L}, bcl-x_{S}, bcl-W, McL-1, A1, NR-13, Ced-9, E1B 19K, BHRF1, KSHV ORF 16, LMW5-HL, KS-bcl-2, etc.), Bax, Bak, Bok, Bad, Bik, Bid (Genbank NM 001196), Blk, Hrk, BNIP3, Bim_{L}, EGL-1, Inhibitors of Apoptosis (e.g., baculovirus OpIAP, DIAP 1 & 2-Drosophila, c-IAP 1 & 2-human, X-IAP-human, NIAP-human, survivin-human, etc.), and the like (see Table I below for an exemplary listing and Genbank Accession Nos.). Given the disclosure provided herein, a desired apoptotic pathway gene can be isolated from a variety of cell types and engineered into an expression vector suitable for use in plants.

TABLE I
Anti- and Pro-Apoptoic Proteins and their Genbank Accession No.

| Anti-apoptotic proteins | Genbank Accession No. |
|---|---|
| Bcl-2 family | |
| Human | |
| Bcl-2 | M14745 |
| Bcl-xL | Z23115, L20121 |
| Mcl-1 | L08246 |
| Bfl-1 (A1) | U27467 |
| Bcl-w | U59747 |
| Other organisms | |
| NR-13 | X84418, Q90343 |
| Ced-9 C. elegans | L26545 |
| Ced-9 C. briggsae | L26546 |
| Viral Bcl-2 homologues | |
| E1B 19K | L19443, AF099665 etc. |
| EBV-BHRF1 | A22899 |
| KSHV ORF 16 | U93872 |
| LMW5-HL | L09548 |
| LMH-5W | Q07818, Q07819 |
| EAR_ASFB7 | P42485 |
| A9 | AAC58120 |
| Cellular IAPs | |
| Human | |
| x-IAP | U32974, NM001167 |
| cIAP-1 | U45878 |
| cIAP-2 | U45879, NP001157 |
| NAIP | Q13075 |
| Survivin | U75285, NP001159 |
| Other organisms | |
| dIAP-1 D. melanogaster | Q24306 |
| dIAP-2 D. melanogaster | U45881, Q24307 |
| BRUCE | CAA76720 |
| Viral IAPs | |
| IAP3 NPVOP | P41437 |
| CfMNPV IAP | AAD00537 |
| GVCP IAP | P41436 |
| BSNPV-IAP | AAC34373 |
| NPVOP-IAP1 | O10296 |
| NPVAC-IAP1 | P41435 |
| NPVEP-IAP | AAD19698 |
| IAP1AcMNPV orf27 IAP1 | AAC63701 |
| NPVLD-IAP | AAC70325 |
| FLIPS | |
| Human | |
| FLICE-like inhibitory protein long form | U97074 |
| Casper | AF010127 |
| FLAME-1-gamma | AF009618 |
| MRIT-alpha-1 | U85059 |
| I-FLICE | AF041458 |
| Usurpin-alpha | AF015450 |
| Other Organisms | |
| FLICE-like inhibitory (mouse) | U97076 |
| CASH alpha protein (mouse) | Y14041 |
| CASH beta protein (mouse) | Y14042 |
| Viral FLIPS | |
| MC160L | U60315 |
| MC159L | U60315 |
| Hypothetical protein E8 | U20824 |

| Pro-apoptotic proteins | Genbank Accession No. |
|---|---|
| Bcl-xS | Z23116, L20122 |
| Bax | L22473 |
| Bak | X84213, U16811, U23765 |
| Bok | AF027954, AF051093 |
| Bad | AF031523 |
| Bik | U34584 |
| Nbk | U49730 |
| Bid | AF042083, NP001187 |
| Hrk | U76376 |
| Blk | AF048838 |
| Bim | AF032457, AF032458 |
| Nip-3 | AF002697 |
| Egl-1 | AF057309 |
| Nip-3 C. elegans homolog | AAD32265 |
| Boo/Diva | AF102501, AF067660 |
| Apaf-1 | AF013263, AF149794 |
| Caspase-1 | X65019 |
| Caspase-2 | U13021 |
| Caspase-3 | U13737 |
| Caspase-4 | U25804 |
| Caspase-5 | U28015 |
| Caspase-6 | U20536 |
| Caspase-7 | U37448 |
| Caspase-8 | U60520 |
| Caspase-9 | U56390 |
| Caspase-10 | U60519 |
| Caspase-11 | Y13089, P70343 |
| Caspase-12 | Y13090 |
| Caspase-13 | NM 003723 |
| Caspase-14 | AF097874 |

The present invention, as described herein, provides plant cells and plant tissues expressing heterologous apoptotic pathway protein encoding genes. Apoptotic pathway genes may be isolated from either genomic DNA or, preferably, cDNA libraries. Isolation of apoptotic pathway protein encoding genes from genomic DNA or cDNA typically can proceed by, first, generating an appropriate DNA library through techniques for constructing libraries that are known in the art (see Sambrook et al., Molecular Cloning: A Laboratory Manual, Cold Spring Harbor Press, 1989) or purchased from commercial sources (e.g., Clontech, Palo Alto, Calif.). Briefly, cDNA libraries can be constructed in bacteriophage vectors (e.g., λAPII), plasmids, or others, which are suitable for screening, while genomic DNA libraries can be constructed in chromosomal vectors, such as YACs (yeast artificial chromosomes), bacteriophage vectors, such as λEMBL3, λgt11, cosmids, or plasmids.

In one embodiment, known apoptotic pathway nucleic acid sequences may be utilized to design an oligonucleotide hybridization probe suitable for screening genomic or cDNA libraries. Preferably, such oligonucleotide probes are 20-30 bases in length. To facilitate hybridization detection, the oligonucleotide may be conveniently labeled, generally at the 5′ end, with a reporter molecule, such as a radionuclide, (e.g., ³²P), enzymatic label, protein label, fluorescent label, or biotin. Such libraries are then generally plated as phage or colonies, depending upon the vector used. Subsequently, a nitrocellulose or nylon membrane, to which the colonies or phage have been transferred, is probed to identify candidate clones which contain the apoptotic pathway protein encoding gene. Such candidates may be verified as containing apoptotic pathway protein encoding DNA by any of a number of various means including, for example, DNA sequence analysis or hybridization with a second, non-overlapping probe.

Once a library is identified as containing an apoptotic pathway protein encoding gene, the gene can be isolated by amplification. Briefly, when using cDNA library DNA as a template amplification primers are designed based upon known apoptotic pathway protein encoding gene sequences (see, e.g., Table I). Primers for amplification are preferably derived from sequences in the 5′ and 3′ untranslated region in order to isolate a full-length cDNA. The primers preferably have a GC content of about 50% and contain restriction sites to facilitate cloning, and do not have self-complementary sequences nor do they contain complementary sequences at their 3′ end (to prevent primer-dimer formation). The primers are annealed to cDNA or genomic DNA and sufficient amplification cycles are performed to yield a product readily visualized by gel electrophoresis and staining. The amplified fragment is purified and inserted into a vector, such as λgt10 or pBS(M13+), and propagated. Confirmation of the nature of the fragment is obtained by DNA sequence analysis or indirectly through amino acid sequencing of the encoded protein.

Other methods may also be used to obtain an apoptotic pathway protein encoding nucleic acid molecule. For example, such a nucleic acid molecule may be obtained from an expression library by screening with an antibody or antibodies reactive to an apoptotic pathway protein (see, Sambrook, et al. Molecular Cloning: A Laboratory Manual, 2nd Ed., Cold Spring Harbor Laboratory Press, NY, 1989; Ausubel, et al. Current Protocols in Molecular Biology, Greene Publishing Associates and Wiley-Interscience, NY, 1995).

Apoptotic pathway protein encoding genes from a variety of species may be isolated using standard methodologies. For closely related species, the human sequence or portion thereof may be utilized as a probe on a genomic or cDNA library. For example, in the case of a caspase, a fragment that encompasses the catalytic site may be labeled and used as a probe on a library constructed from mouse, primate, rat, dog, or other vertebrate, warm-blooded or mammalian species. An initial hybridization at normal stringency may yield candidate clones or fragments. If no hybridization is initially observed, varying degrees of stringency may be used (see Sambrook et al., supra, and other well-known sources for stringency conditions). While such probes may also be used to probe libraries from evolutionarily diverse species, such as Drosophila, hybridization conditions will likely be more relaxed.

While relaxed hybridization conditions using probes designed from human sequences may identify apoptotic pathway protein encoding genes of evolutionarily diverse species it may be more beneficial to attempt to directly isolate these genes from a library using methods which do not require the human sequence per se. These methods include, but are not limited to, amplification using primers derived from conserved areas, amplification using degenerate primers from various regions, antibody probing of expression libraries, and the like. For example, random-primed amplification (e.g., polymerase chain reaction) may be employed (see, e.g., Methods Enzymol. 254:275, 1995; Trends Genet. 11:242, 1995; Liang and Pardee, Science 257:967, 1992; Welsh et al., Nucl. Acids Res. 20:4965, 1992). In addition, variations of random-primed PCR may also be used, especially when a particular gene or gene family is desired. In such a method, one of the amplification primers is an “anchored oligo(dT) (oligo(dT)dN)” and the other primer is a degenerate primer based upon amino acid or nucleotide sequence of a related gene. A gene sequence is identified as an apoptotic pathway protein by amino acid similarity, nucleic acid similarity, and/or functional equivalence. Further, a gene may be identified as an apoptotic pathway protein based on computer algorithms that compare functionality and secondary structure or relate function to domain structure in a tertiary format, such algorithms are currently available. Candidate genes are expressed and the gene product is examined for enzyme activity and/or other functional activities using standard apoptotic assays and assays described herein or other equivalent assays.

Variants of apoptotic pathway protein encoding genes provided herein may be engineered from natural variants (e.g., polymorphisms, splice variants, mutants), synthesized or constructed. Many methods have been developed for generating mutants (see, generally, Sambrook et al., supra; Ausubel, et al., supra, and the discussion above). Briefly, preferred methods for generating a few nucleotide substitutions utilize an oligonucleotide that spans the base or bases to be mutated and contains the mutated base or bases. The oligonucleotide is hybridized to complementary single stranded nucleic acid and second strand synthesis is primed from the oligonucleotide. The double-stranded nucleic acid is prepared for transformation into host cells, typically E. coli, but alternatively, other prokaryotes, yeast or other eukaryotes. Standard screening and vector growth protocols are used to identify mutant sequences and obtain high yields.

Similarly, deletions and/or insertions of the apoptotic pathway protein encoding genes may be constructed by any of a variety of known methods as discussed supra. For example, the gene can be digested with restriction enzymes and religated such that a sequence is deleted or religated with additional sequences such that an insertion or large substitution is made. Other means of generating variant sequences may be employed with methods known in the art, for example those described in Sambrook et al., supra and Ausubel et al., supra. Verification of variant sequences is typically accomplished by restriction enzyme mapping, sequence analysis, or probe hybridization.

Transgenic plants and plant cells expressing antisense and ribozyme molecules directed to plant apoptotic pathway proteins are also encompassed within the scope of the present invention. The expression of antisense RNA molecules will act to directly block the translation of mRNA by binding to targeted mRNA and preventing protein translation. The expression of ribozymes, which are enzymatic RNA molecules capable of catalyzing the specific cleavage of RNA may also be used to block protein translation. The mechanism of ribozyme action involves sequence specific hybridization of the ribozyme molecule to complementary target RNA, followed by an endonucleolytic cleavage. Within the scope of the invention are engineered hammerhead motif ribozyme molecules that specifically and efficiently catalyze endonucleolytic cleavage of RNA sequences. RNA molecules may be generated by transcription of DNA sequences encoding the RNA molecule.

Nucleic acids and oligonucleotides for use as described herein can be synthesized by any method known to those of skill in this art (see, e.g., WO 93/01286, U.S. application Ser. No. 07/723,454; U.S. Pat. No. 5,218,088; U.S. Pat. No. 5,175,269; U.S. Pat. No. 5,109,124). Identification of ribozymes and oligonucleotides for use as antisense agents, and DNA for genetic therapy involve methods well known in the art. For example, the desirable properties, lengths and other characteristics of such oligonucleotides are well known.

Antisense nucleotides are oligonucleotides that bind in a sequence-specific manner to nucleic acids, such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye; U.S. Pat. No. 5,135,917 to Burch; U.S. Pat. No. 5,087,617 to Smith and Clusel et al., Nucl. Acids Res. 21:3405-3411, 1993, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

Particularly useful antisense nucleotides and triplex molecules are molecules that are complementary to or bind the sense strand of DNA or mRNA that encodes a protein involved in modulating the apoptosis pathway or a protein mediating any other unwanted process such that inhibition of translation of the protein is desirable.

Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., Tetrehedron Lett. 28:3539-3542, 1987; Miller et al., J. Am. Chem. Soc. 93:6657-6665, 1971; Stec et al., Tetrehedron Lett. 26:2191-2194, 1985; Moody et al., Nucl. Acids Res. 12:4769-4782, 1989; Uznanski et al., Nucl. Acids Res., 1989; Letsinger et al., Tetrahedron 40:137-143, 1984; Eckstein, Annu. Rev. Biochem. 54:367-402, 1985; Eckstein, Trends Biol. Sci. 14:97-100, 1989; Stein, in: Oligodeoxynucleotides. Antisense Inhibitors of Gene Expression, Cohen, Ed, Macmillan Press, London, pp. 97-117, 1989; Jager et al., Biochemistry 27:7237-7246, 1988).

With regard to apoptotic pathway proteins produced in plants, such proteins can be isolated by standard methods, such as affinity chromatography, size exclusion chromatography, metal ion chromatography, ionic exchange chromatography, HPLC, and other known protein isolation methods. (see generally Ausubel et al., supra; Sambrook et al., supra). An isolated purified protein gives a single band on SDS-PAGE when stained with Coomassie blue. It should be recognized the present invention encompasses production of endogenous plant apoptotic pathway proteins as well as the recombinant production of non-plant apoptotic proteins, such as mammalian apoptotic proteins, thereby using the plant as a recombinant production vehicle. In addition, the present invention may also be used to study animal genes and their functionality in a plant cell. Accordingly, as described in greater detail below, by generating a transgenic plant harboring an apoptotic pathway protein that inhibits apoptosis, recombinant production of any number of peptides and polypeptides, including non-apoptosis related peptides and polypeptides, may be greatly enhanced by reducing cell death events associated with recombinant production of endogenous or heterologous polypeptides (e.g., antibodies).

Apoptotic pathway proteins may be expressed as a hexa-his fusion protein and isolated by metal-containing chromatography, such as nickel-coupled beads. Briefly, a sequence encoding His₆ is linked to a DNA sequence encoding an apoptotic pathway protein. Although the His₆ sequence can be positioned anywhere in the molecule, preferably it is linked at the 3′ end immediately preceding the termination codon. The fusion may be constructed by any of a variety of methods. A convenient method is amplification of the apoptotic pathway protein encoding gene using a downstream primer that contains the codons for His₆.

Purified apoptotic pathway proteins may be used in assays to screen for inhibitory compounds. These assays may be performed in vitro or in vivo and utilize any of the methods described herein or as known in the art. The protein may also be crystallized and subjected to X-ray analysis to determine its 3-dimensional structure, or used to raise antibodies. In addition, as described, infra, heterologous nucleic acids encoding apoptotic pathway proteins can be expressed in plants to create plant cells to be used in screening assays for inhibitors and enhancers of apoptosis

B. Vectors and Transformation Methodologies

Apoptotic pathway proteins may be expressed in a variety of host organisms. However, as noted herein these proteins offer unexpected advantages when placed within plant cells, particularly where no similar apoptotic pathway proteins have been identified. For example, when expressed in plant cells apoptotic pathway inhibitor proteins may be used to increase biotic and abiotic insult resistance, including resistance to fungal and viral infections. Further, the ability to modulate cell death in plants offers the ability to couple expression of insecticidal compounds, for example, Bacillus thuringiensis encoded toxins (see e.g., PCT Application WO 97/34926), within the plant. Accordingly, higher insecticidal tolerance within the plant can be achieved in a plant that would normally initiate a programmed cell death cycle when expressing certain types or levels of insecticidal compounds. Similarly, higher herbicide tolerance can be achieved by increasing the plant cell's resistance to programmed cell death events induced by herbicides. Transfer of genetic material is routine in many plant types and possible to varying degrees in all plant types. One skilled in the art will appreciate that there are a wide variety of suitable vectors for expression in plant cells readily obtainable.

1. Vectors

Transgenic plants of the present invention are produced by contacting a plant cell with a vector comprising a heterologous nucleic acid sequence comprising at least one structural gene encoding a protein that modulates programmed cell death (i.e., apoptosis). To be effective once introduced into plant cells, the structural gene of interest should be operably associated with a promoter which is effective in the plant cells to cause transcription of the gene of interest. Additionally, a polyadenylation sequence or transcription control sequence, also recognized in plant cells, may be employed. It is preferred that the vector harboring the heterologous nucleic acid sequence also contain one or more selectable marker genes so that the transformed cells can easily be selected from non-transformed cells in culture, as described herein.

Those of ordinary skill in the art will be able to select an appropriate vector for introduction of a heterologous nucleic acid sequence in a relatively intact state. Thus, any vector that will produce a plant carrying the introduced DNA sequence is sufficient. Accordingly, naked DNA can be utilized even though only low efficiency transformation will likely occur. The selection of the vector, or whether to use a vector, is typically guided by the method of transformation selected.

In brief, a DNA sequence encoding an apoptotic pathway protein is introduced into an expression vector appropriate for the host plant cell. In certain embodiments, the apoptotic pathway protein is an inhibitor of apoptosis, while in other embodiments, the apoptotic pathway protein is an enhancer or initiator of apoptosis. Preferred inhibitors include, but are not limited to IAP, Bcl-2, BCI-X_{L}, Bcl-W, McL-1, A1, NR13, CED-9, E1B 19K, BHRF1, Bag-1, and the like. In other embodiments, the apoptotic pathway protein encoding nucleic acid is inserted into a vector and linked to another polypeptide encoding nucleic acid molecule such that a fusion protein is produced. The apoptotic pathway protein sequence is derived, as described herein. As discussed above, the sequence may contain alternative codons for each amino acid with multiple codons. The alternative codons can be chosen as “optimal” for the host species, such optimization protocols are well known in the art. Further, restriction sites are typically incorporated into the primer sequences and are chosen with regard to the cloning site of the vector. If necessary, translational initiation and termination codons can be engineered into the primer sequences.

At a minimum, the vector should contain a promoter sequence. As noted above, a “promoter” refers to a nucleotide sequence that contains elements that direct the transcription of a linked/associated gene. At a minimum, a promoter contains an RNA polymerase binding site. More typically, in eukaryotes, promoter sequences contain binding sites for other transcriptional factors that control the rate and timing of gene expression. Such sites include TATA box, CAAT box, POU box, AP1 binding site, and the like. Promoter regions may also contain enhancer elements. When a promoter is linked to a gene so as to enable transcription of the gene, it is “operatively linked or associated” therewith.

Other regulatory sequences may also be included. Such sequences include a transcription termination signal sequence, introns, secretion signal sequence, origin of replication, selectable marker, and the like. The regulatory sequences are operably associated with one another to allow transcription or translation.

The expression vectors used herein include a promoter designed for expression of the proteins in a plant host cell. Suitable promoters are widely available and are well known in the art. Inducible or constitutive promoters are preferred.

In other preferred embodiments, the vector also includes a transcription terminator sequence. A “transcription terminator region” has either a sequence that provides a signal that terminates transcription by the polymerase that recognizes the selected promoter and/or a signal sequence for polyadenylation.

As noted above, vector(s) employed in the present invention generally comprise a nucleic acid sequence comprising at least one structural gene encoding an apoptotic pathway protein, operably associated with a promoter. To commence a transformation process in accordance with the present invention, it is first necessary to construct a suitable vector and properly introduce it into the plant cell. The details of the construction of the vectors utilized herein are known to those skilled in the art of plant genetic engineering as are the requisite transformation methods, however, both are discussed in greater detail herein. Typically an expression vector contains prokaryotic nucleic acid elements coding for a bacterial replication origin (e.g., E. coli ori) and a reporter gene or a selectable marker (e.g., antibiotic resistance) to provide for the growth and selection of the expression vector in a bacterial host; nucleic acid elements that control initiation of transcription, such as a promoter for both plant and bacterial growth; nucleic acid elements that control the processing of transcripts such as transcription termination/polyadenylation sequence; and a reporter gene or marker that is operably associated with a promoter. Useful reporter genes include β-glucuronidase, β-galactosidase, chloramphenicol acetyl transferase, luciferase, and the like. Preferably the reporter gene is either β-glucuronidase (GUS) or green fluorescent protein (GFP). Preferable selectable markers include antibiotic resistance such as G418, hygromycin, kanamycin, bialophos (imparted by the bar gene), etc. In certain embodiments, bacterial expression systems are utilized to synthesize amounts of a construct for subsequent restriction endonucleases digestion to excise the gene of interest. The gene of interest is then ligated into a plant expression vector and used to transform plant cells. Accordingly, one skilled in the art will readily recognize that one may utilize entirely different reporter genes or selectable markers in bacteria as opposed to plants. Preferred bacterial selection is performed using ampicillin resistance, while transgenic plant cells are typically selected based on kanamycin resistance.

General descriptions of plant expression vectors and reporter genes can be found in Gruber et al., “Vectors for Plant Transformation, in Methods in Plant Molecular Biology & Biotechnology” in Glich et al., Eds. pp. 89-119, CRC Press, 1993. Moreover, GUS expression vectors and GUS gene cassettes are available from Clontech Laboratories, Inc., Palo Alto, Calif. while GFP expression vectors and GFP gene cassettes are available from Aurora Biosciences. (San Diego, Calif.).

The nucleic acid sequence encoding apoptotic pathway proteins may also include a secretion signal, whereby the resulting polypeptide is a precursor protein processed and secreted. The resulting processed protein may be recovered from the plant cell or tissues or phloem. Secretion signals suitable for use are widely available and are well known in the art, see e.g., During et al., Plant Mol. Biol. 15:281-293, 1990; Lindsey and Jones in: Plant Cell Line Selection, pp. 317-335, VCH Weinham, Germany, 1990; Gruber and Crosby in: Methods in Plant Molecular Biology and Biotechnology, pp. 89-117, CRC Press, Boca Raton, Fla., 1993.

The heterologous nucleic acid sequences utilized in the present invention can be introduced into plant cells using Ti plasmids, root-inducing (Ri) plasmids, particle bombardment, electroporation, and plant virus vectors. (For reviews of such techniques and vectors, see, for example, Weissbach & Weissbach, Methods for Plant Molecular Biology, Academic Press, NY, Section VHI, pp. 421-463; 1988; Grierson & Corey, Plant Molecular Biology, 2d Ed., Blackie, London, Ch. 7-9, 1988, and Horsch et al., Science 227:1229, 1985; and Gene Transfer to Plants, eds. Potrykus. Springer Verlaa, 1995) all incorporated herein by reference.

Expression vectors containing genomic or synthetic fragments can be introduced into protoplast or into intact tissues or isolated cells. Preferably expression vectors are introduced into intact tissue. General methods of culturing plant tissues are provided for in Miki et al., in Methods in Plant Molecular Biology & Biotechnology, Glich et al. Eds., pp. 67-88 CRC Press, 1993); and by Phillips et al. in Corn & Corn Improvement, 3rd Edition, Sprague et al., Eds. American Society of Agronomy Inc. et al. pp. 345-387 1988 and as described in more detail below.

2. Promoters

Once the host plant has been selected and the method of gene transfer into the plant determined, a constitutive, a inducible/developmentally regulated, or a tissue specific promoter for the host plant is selected so that the foreign protein is expressed in the desired part(s) of the plant (e.g., all cells, some cells, or whole tissues). As those of ordinary skill in the art will readily appreciate, any plant compatible promoter may be used to drive the expression of a structural gene and, more specifically, an apoptotic pathway gene. Although the endogenous promoter of the structural gene of interest may be utilized for transcriptional regulation of the gene, preferably, the promoter is a foreign regulatory sequence. For plant expression vectors, suitable viral promoters include the 35S RNA and 19S RNA promoters of CaMV (Brisson et al., Nature 310:511, 1984; Odell et al., Nature 313:810, 1985); the full-length transcript promoter from Figwort Mosaic Virs FMV) (Gowda et al., J. Cell Biochem. 13D: 301, 1989 and U.S. Pat. No. 5,378,619) and the coat protein promoter to TMV (Takamatsu et al., EMBO J 6:307, 1987). Alternatively, plant promoters such as the light-inducible promoter from the small subunit of ribulose bis-phosphate carboxylase (ssRUBISCO) (Coruzzi et al., EMBO J 3:1671, 1984; Broglie et al., Science 224:838, 1984); mannopine synthase promoter (Velten et al., EMBO J 3:2723, 1984) nopaline synthase (NOS) and octopine synthase (OCS) promoters (carried on tumor-inducing plasmids of Agrobacterium tumefaciens) or heat shock promoters, e.g., soybean hsp17.5-E or hsp17.3-B (Gurley et al., Mol. Cell. Biol. 6:559, 1986; Severin et al., Plant Mol. Biol. 15:827, 1990) may be used. See PCT Publication WO 91/19806 for a review of a variety of known plant promoters which are suitable for use within the context of the present invention.

a. Constitutive Promoters

Promoters useful within the context of the present invention include both constitutive and inducible natural promoters as well as engineered promoters. Such promoters may be obtained from plants, viruses, or other sources, and include, but are not limited to: the 35S promoter of cauliflower mosaic virus (CaMV), as used herein, the phrase “CaMV 35S” promoter includes variations and analogs of the CaMV 35S promoter, e.g., promoters derived by means of ligations with operator regions, random or controlled mutagenesis, tandem promoters, etc.; promoters of seed storage protein genes such as Zma10 Kz or Zmag12 (maize zein and glutelin genes, respectively), or “housekeeping genes” that express in all cells (such as Zmaact, a maize actin gene). (see, Benfey et al., Science 244:174-181, 1989; Elliston in Plant Biotechnology, eds. Kung and Arntzen, Butterworth Publishers, Boston, Mass., p. 115-139, 1989). Accordingly, the present invention can utilize promoters for genes which are known to give high expression in edible plant parts, such as the patatin gene promoter from potato, see e.g., Wenzler et al., Plant Mol. Biol. 12:41-45, 1989. While the CaMV promoters are common examples of constitutive promoters so are the ubiquitin promoter (see EP Patent Application 0342926) and the Chlorella virus DNA methyltransferase promoter (see U.S. Pat. No. 5,563,328).

b. Inducible Promoters

As noted above, inducible promoters are useful within the context of the present invention. An inducible promoter is a promoter that is capable of directly or indirectly activating transcription of a nucleic acid sequence in response to an inducer. In the absence of an inducer, the sequence will not be transcribed. In one embodiment, the protein factor that binds specifically to an inducible promoter to activate transcription is present in an inactive form which is then directly or indirectly converted to the active form by the inducer. The inducer may be a biotic or abiotic insult such as, a chemical agent such as a protein, metabolite (sugar, alcohol, etc.), a growth regulator, herbicide, or a phenolic compound or a physiological stress imposed directly by heat, salt, toxic elements, etc. or indirectly through the action of a pathogen or disease agent such as a virus. A plant cell containing an inducible promoter may be exposed to an inducer by externally applying the inducer to the cell such as by spraying, watering, heating, exposure to light, exposure to a pathogen, or similar methods. In certain embodiments, inducible promoter may be in an induced state throughout seed formation or at least for a period which corresponds to the transcription of the nucleic acid sequence of the recombinant nucleic acid molecule(s).

To be most useful, an inducible promoter preferably provides low or no expression in the absence of the inducer; high expression in the presence of the inducer; uses an induction scheme that does not interfere with the normal physiology of the plant; and has little effect on the expression of other genes. Examples of inducible promoters useful within the context of the present invention include those induced by chemical means, such as the yeast metallothionein promoter which is activated by copper ions (Mett et al., Proc. Natl. Acad. Sci. U.S.A. 90:4567, 1993); In2-1 and In2-2 regulator sequences which are activated by substituted benzenesulfonamides, e.g., herbicide safeners (Hershey et al., Plant Mol. Biol 17:679, 1991); the chemically inducible gene promoter sequence isolated from a 27 kD subunit of the maize glutathione-S-transferase (GST II) gene, induced by are N,N-diallyl-2,2-dichloroacetamide (common name: dichloramid) or benzyl-2-chloro-4-(trifluoromethyl)-5-thiazolecarboxylate (common name: flurazole) and as described in published PCT Publication No. WO 90/08830; the GRE regulatory sequences which are induced by glucocorticoids (Schena et al., Proc. Natl. Acad. Sci. USA. 88:10421, 1991; 70 kDa heat shock promoter of D. melanogaster (Freeling and Bennet, Maize ADN 1, Ann. Rev. of Genetics 19:297-323) and the alcohol dehydrogenase promoter which is induced by ethanol (Nagao et al. Miflin, Ed, Oxford Surveys of Plant Molecular and Cell Biology, Vol. 3, p. 384-438, Oxford University Press, Oxford, 1986) or the Lex A promoter which is triggered with chemical treatment and is available through Ligand Pharmaceuticals. Other inducible promoters include those induced by pathogen attack, such as nematode-inducible promoters described in PCT Publication number WO 98/22599, the glucocorticoid inducible promoter (Aoyama and Chua, The Plant J. 11:605-612, 1997), a chalcone synthase promoter, and the defense activated promoter (prop1-1) (Strittmatter et al., Bio/Technology 13:1085-1089, 1995). Inducible promoters have also been described in published Application No. EP89/103888.7 by Ciba-Geigy, wherein a number of inducible promoters are identified, including the PR protein genes, especially the tobacco PR protein genes, such as PR-1a, PR-1b, PR-1c, PR-1, PR-A, PR-S, the cucumber chitinase gene, and the acidic and basic tobacco beta-1,3-glucanase genes. Wound inducible (WIN) promoters may also be useful in the context of the present invention, see Lindsey and Jones in: Plant Cell Line Selection, pp. 317-335, VCH Weinham, Germany, 1990, for a discussion thereon. Other promoters, both constitutive and inducible and enhancers are known to those of ordinary skill in the art.

c. Tissue Specific Promoters

Tissue specific promoters may also be utilized in the present invention. An example of a tissue specific promoter is the promoter expressed in shoot meristems (Atanassova et al., Plant J. 2:291, 1992). Other tissue specific promoters useful in transgenic plants, including the cdc2a promoter and cyc07 promoter, will be known to those of skill in the art. (See, for example, Ito et al., Plant Mol. Biol. 24:863, 1994; Martinez et al., Proc. Natl. Acad. Sci. USA 89:7360, 1992; Medford et al., Plant Cell 3:359, 1991; Terada et al., Plant Journal 3:241, 1993; Wissenbach et al., Plant Journal 4:411, 1993; tuber-directed class I patatin promoter, Bevan et al., Nucleic Acids Res. 14:4625-38, 1986; the promoters associated with potato tuber ADPGPP genes, Muller et al., Mol. Gen. Genet. 224:136-46, 1990; the soybean promoter of β-conglycinin, also known as the 7S protein, which drives seed-directed transcription, Bray, Planta 172:364-370, 1987; seed-directed promoters from the zein genes of maize endosperm, Pedersen et al., Cell 29:1015-26, 1982; and pollen specific promoters such as those disclosed in U.S. Pat. No. 5,086,169 to Mascarenhas which discloses a pollen specific promoter isolated from maize; U.S. Pat. No. 5,412,085 to Allen et al., which discloses another pollen specific promoter from maize; U.S. Pat. No. 5,477,002 to Tuttle et al., which discloses an anther specific promoter; U.S. Pat. No. 5,470,359 to Huffman et al., which discloses a tapetum specific promoter; WO 92/11379 to Draper et al. which discloses a tapetum specific promoter isolated from Brassicaceae, Plant 8(1):55-63, 1995, which discloses a pollen specific promoter from tobacco.

It may also be desirable to include intron sequences in the promoter constructs since the inclusion of intron sequences in the coding region may result in enhanced expression and specificity. Thus, it may be advantageous to join the DNA sequences to be expressed to a promoter sequence that contains the first intron and exon sequences of a polypeptide which is unique to cells/tissues of a plant. Additionally, regions of one promoter may be joined to regions from a different promoter in order to obtain a chimeric promoter.

3. Markers

The vectors of the present invention, also preferably include at least one selectable or scorable marker/reporter that is functional in the host. Numerous genes for this purpose have been identified. (see, for example, Fraley, in Plant Biotechnology, eds. Kung and Arntzen, Butterworth Publishers, Boston, Mass., p. 395-407, 1989; Weising et al., Ann. Rev. Genet. 22:421-477, 1988). A selectable marker gene includes any gene that confers a phenotype or trait on the host that allows transformed cells to be identified and selectively grown. Accordingly, the selection marker genes encode a selection gene product which confers on a plant cell resistance to a chemical agent or physiological stress, or confers a distinguishable phenotypic characteristic to the cells such that plant cells transformed with the recombinant nucleic acid molecule may be easily selected using a selective agent. Suitable selectable marker genes for bacterial hosts include the ampicillin resistance gene (Amp^{r}), tetracycline resistance gene (Tc^{r}) and the kanamycin resistance gene (Kan^{r}). The ampicillin resistance gene is presently preferred. Suitable markers for eukaryotes usually require a complementary deficiency in the host (e.g., thymidine kinase (tk) in tk-hosts). However, drug markers are also available (e.g., G418 resistance and hygromycin resistance). Examples of suitable selectable markers include, the bar gene, adenosine deaminase, dihydrofolate reductase, hygromycin-B-phosphotransferase, thymidine kinase, xanthine-guanine phosphoribosyl-transferase, and amino-glycoside 3′-O-phosphotransferase II (kanamycin, neomycin, and G418 resistance). Other suitable markers are known to those of skill in the art. In addition, identification of gene expression can be performed by such methods as Northern Blots of the mRNA of interest produced by the plant.

4. Transformation Methods

The transformation of plants in accordance with the invention may be carried out in essentially any of the various ways known to those skilled in the art of plant molecular biology. (See, for example, Methods of Enzymology, Vol. 153, 1987, Wu and Grossman, Eds., Academic Press, incorporated herein by reference). As used herein, the term “transformation” means alteration of the genotype of a host plant by the introduction of a heterologous nucleic acid sequence.

Methods of introducing expression vectors into plant tissue include physical and chemical means, including electroporation, particle bombardment, viral and bacterial infection/co-cultivation with, for example, Agrobacterium tumefaciens. These methods of transformation are useful for introducing foreign genes into both monocotyledenous and dicotyledenous plants. Potrykus, Annu. Rev. Plant Physiol., Plant Mol. Biol. 42:205-225, 1991, and Shimamoto et al., Nature 338:274-276, 1989. The principle methods of causing stable integration of exogenous DNA into plant genomic DNA include the following approaches: 1) Agrobacterium—mediated gene transfer; Horsch et al., Science 227:1229, 1985; Gruber et al., supra.; Klee et al., Annu. Rev. Plant Physiol. 38:467-486, 1987; Klee et al., Molecular Biology of Plant Nuclear Genes 6:2-25, 1989; Gatenby, Plant Biotechnology, 93-112, 1989; White, Plant Biotechnology, 3-34 1989; and 2) direct DNA uptake, Paszkowski et al., Molecular Biology of Plant Nuclear Genes 6:52-68, 1989, including methods for direct uptake of DNA into protoplasts, Toriyama et. al., Bio/Technology 6:1072-1074, 1988; DNA uptake induced by brief electric shock of plant cells, Zhang et al., Plant Cell Rep. 7:379-384, 1988, and Fromm et al., Nature 319:791-792, 1986. DNA injection into plant cells or tissues by particle bombardment, Klein et al., Progress in Plant Cellular and Molecular Biology, 56-66, 1988; Klein et al., Bio/Technology 6:559-563, 1988; McCabe et al., Bio/Technology 6:923-926, 1988, and Sanford, Physiol. Plant 79:206-209, 1990, by the use of micropipette systems, Hess, Int. Rev. Cytol 107:367-395, 1987, Neuhaus et al., Theor. Appl Genet. 75:30-36, 1987, Neuhaus and Spangenberg, Physiol. Plant. 79:213-217, 1990, or by the direct incubation of DNA with germinating pollen; DeWet et al., Experimental Manipulation of Ovule Tissue, 197-209, 1985, Ohta, Y. Proc. Natl. Acad. Sci USA 83.715-719, 1986; or 3) the use of plant virus as gene vectors, Klee et al., Ann. Rev. Plant Physiol. 38:467-486, 1987; Futterer et al., Physiol. Plant 79:154-157, 1990.

The use of Cauliflower Mosaic Virus (CaMV) (Howell et al., Science 208:1265, 1980) and gemini viruses (Goodman, J. Gen Virol. 54:9, 1981) as vectors has been suggested but by far the greatest reported successes have been with Agrobacteria sp. (Rorsch et al., Science 227:1229-1231, 1985). Methods for the use of Agrobacterium based transformation systems have now been described for many different species. Generally strains of bacteria are used that harbor modified versions of the naturally occurring Ti plasmid such that DNA is transferred to the host plant without the subsequent formation of tumors. These methods involve the insertion within the borders of the Ti plasmid the DNA to be inserted into the plant genome linked to a selection marker gene to facilitate selection of transformed cells. Such a vector system is described by Hajdukiewicz et al., Plant Mol. Bio. 25:989-994, 1994. Such a binary system comprises a first Ti plasmid having a virulence region (vir) essential for the introduction of transfer DNA (T-DNA) into plants, and a chimeric plasmid. The latter contains at least one border region of the T-DNA region of a wild-type Ti plasmid flanking the nucleic acid to be transferred. Binary Ti plasmid systems have been shown effective to transform plant cells (De Framond Biotechnology 1:262, 1983; Hoekema et al., Nature 303:179, 1983). Additional binary systems are described in PCT Publication Nos. WO 99/01563 and WO 99/10514.

Bacteria and plant tissues are cultured together to allow transfer of foreign DNA into plant cells then transformed plants are regenerated on selection media. Any number of different organs and tissues can serve as targets from Agrobacterium mediated transformation as described specifically for members of the Brassicaceae. These include thin cell layers (Charest et al., Theor. Appl Genet. 75:438-444, 1988), hypocotyls (DeBlock et al., Plant Physiol. 91:694-701, 1989), leaf discs (Feldman, Plant Sci. 47:63-69, 1986), stems (Fry et al., Plant Cell Repts. 6:321-325, 1987), cotyledons (Moloney et al., Plant Cell Repts 8:238-242, 1989) and embryoids (Neuhaus et al., Theor. Appl. Genet. 75:30-36, 1987). One skilled in the art understands, however, that it may be desirable in some crops to choose a different tissue or method of transformation.

It may be useful to generate a number of individual transformed plants with any recombinant construct in order to recover plants free from any position effects. In certain embodiments it may be preferable to select plants that contain one copy of the introduced heterologous nucleic acid molecule in order to minimize silencing effects.

In one embodiment, expression vectors are introduced into plant tissues using co-cultivation of plant cells with viruses or bacterium. For example, plant RNA viral based systems can used, typically by inserting the structural gene of interest into the coat promoter regions of a suitable plant virus under the control of a promoter. Plant RNA viral based systems are described, for example, in U.S. Pat. Nos. 5,500,360; 5,316,931, and 5,589,367, each of which is incorporated herein by reference in its entirety.

As noted above, in certain embodiments of the present invention, the Agrobacterium—Ti plasmid system is utilized, Watson et al., Recombinant DNA, a Short Course, Scientific American Books, 164-175, 1983. The tumor-inducing (Ti) plasmids of A. tumefaciens contain a segment of plasmid DNA known as transforming DNA (T-DNA) which is transferred to plant cells where it integrates into the plant host genome. The construction of the transformation vector system has two basic steps. First, a plasmid vector is constructed which replicates in Escherichia coli (E. coli.). This plasmid contains the DNA encoding the protein of interest (an antigenic protein in this invention); this DNA is flanked by T-DNA border sequences that define the points at which the DNA integrates into the plant genome. Usually a gene encoding a selectable marker (such as a gene encoding resistance to an antibiotic such as Kanamycin) is also inserted between the left border (LB) and right border (RB) sequences; the expression of this gene in transformed plant cells gives a positive selection method to identify those plants or plant cells which have an integrated T-DNA region, Watson et al., Recombinant DNA, a Short Course, Scientific American Books, 164-175, 1983; White, Plant Biotechnology, 3-34, 1989. The second step entails transfer of the plasmid from E. coli to Agrobacterium. This can be accomplished via a conjugation mating system, or by direct uptake of plasmid DNA by Agrobacterium. For subsequent transfer of the T-DNA to plants, the Agrobacterium strain utilized contains a virulence (vir) genes for T-DNA transfer to plant cells, White, Plant Biotechnology, 3-34, 1989; Fraley, Plant Biotechnology, 395-407, 1989; Hajdukiewicz et al., Plant Mol. Bio. 25:989-994, 1994. Those skilled in the art should recognize that there are multiple choices of Agrobacterium strains and plasmid construction strategies that can be used to optimize genetic transformation of plants. They will also recognize that A. tumefaciens may not be the only Agrobacterium strain used. Other Agrobacterium strains such as A. rhizogenes might be more suitable in some applications. Methods of inoculation of the plant tissue vary depending upon the plant species and the Agrobacterium delivery system. A very convenient approach is the leaf disc procedure which can be performed with any tissue explant that provides a good source for initiation of whole plant differentiation. The addition of nurse tissue may be desirable under certain conditions. Other procedures such as the in vitro transformation of regenerating protoplasts with A. tumefaciens may be followed to obtain transformed plant cells as well, Potrykus, Plant Mol. Biol. 42:205-225, 1991; White, Plant Biotechnology, 3-34, 1989.

Methods involving the use of Agrobacterium include, but are not limited to: 1) co-cultivation of Agrobacterium with cultured isolated protoplasts; 2) transformation of plant cells or tissues with Agrobacterium; or 3) transformation of seeds, apices or meristems with Agrobacterium. In addition, gene transfer can be accomplished by in situ transformation by Agrobacterium, as described by Bechtold et al., C. R. Acad. Sci. Paris 316:1194, 1993 and transformation methods as described in U.S. patent application Ser. No. 08/667,188 and corresponding PCT Publication No. WO 97/48814, and exemplified herein. In brief, this approach is based on the vacuum infiltration of a suspension of Agrobacterium cells.

The preferred method of introducing heterologous nucleic acid into plant cells is to infect such plant cells, an explant, a meristem or a seed, with transformed Agrobacterium tumefaciens as described above. Under appropriate conditions known in the art, the transformed plant cells are grown to form shoots, roots, and develop further into plants. In one embodiment, vector(s) of the invention comprises a Ti plasmid binary system wherein the heterologous nucleic acid sequence encodes an apoptotic pathway protein. In certain embodiments, the nucleic acid sequences encode at least one anti-apoptotic protein. Such vectors may optionally contain a nucleic acid sequence which encodes a second or more apoptotic pathway protein. Alternatively, two or more vectors can be utilized wherein each vector contains a heterologous nucleic acid sequence. Other apoptotic pathway genes can be utilized for construction of one or more vectors, in a similar manner.

In certain embodiments, heterologous nucleic acid sequences can be introduced into a plant cell by contacting the plant cell using direct physical or chemical means. For example, the nucleic acid can be physically transferred by microinjection directly into plant cells by use of micropipettes or particle bombardment. Alternatively, the nucleic acid may be transferred into the plant cell by using polyethylene glycol which forms a precipitation complex with genetic material that is taken up by the cell.

Another method for introducing nucleic acid into a plant cell is high velocity ballistic penetration by small particles with the nucleic acid to be introduced contained either within the matrix of small beads or particles, or on the surface thereof (Klein et al., Nature 327:70, 1987; U.S. Pat. Nos. 4,945,050, 5,036,006, and 5,100,792). Typically, when utilizing small particle bombardment, the DNA is adsorbed on microprojectiles such as magnesium sulfate crystals or tungsten particles, and the microprojectiles are physically accelerated into cells or plant tissues. Although, typically only a single introduction of a new nucleic acid sequence is desired, this method typically provides for multiple introductions.

Heterologous nucleic acid can also be introduced into plant cells by electroporation (Fromm et al., Proc. Natl. Acad. Sci., U.S.A. 82:5824, 1985, which is incorporated herein by reference). In this technique, plant protoplasts are electroporated in the presence of vectors or nucleic acids containing the relevant nucleic acid sequences. Electrical impulses of high field strength reversibly permeabilize membranes allowing the introduction of nucleic acids. Electroporated plant protoplasts reform the cell wall, divide and form a plant callus. Selection of the transformed plant cells with the transformed gene can be accomplished using phenotypic markers as described herein.

Further, as described above, cauliflower mosaic virus (CaMV) may also be used as a vector for introducing heterologous nucleic acid into plant cells (U.S. Pat. No. 4,407,956). CaMV viral DNA genome is inserted into a parent bacterial plasmid creating a recombinant DNA molecule which can be propagated in bacteria. After cloning, the recombinant plasmid again may be cloned and further modified by introduction of the desired nucleic acid sequence. The modified viral portion of the recombinant plasmid is then excised from the parent bacterial plasmid, and used to inoculate the plant cells or plants.

In the various embodiments, the invention provides methods for producing a transgenic plant having modulated apoptosis, abiotic insult, and/or biotic insult resistance. The method comprises contacting a plant cell with a vector, comprising a heterologous nucleic acid sequence comprising at least one structural gene encoding an apoptotic pathway protein, operably associated with a promoter, producing a plant from said transformed plant cell; and selecting a plant exhibiting abiotic or biotic insult resistance and/or modulated apoptosis.

As used herein, the term “contacting” refers to any means of introducing the vector(s) into the plant cell, including chemical, physical, or mechanical means as described above. Preferably, contacting refers to introducing the nucleic acid or vector into plant cells (including an explant, a meristem or a seed), via Agrobacterium tumefaciens transformed with the heterologous nucleic acid as described above.

Normally, a plant cell is regenerated to obtain a whole plant from the transformation process. The immediate product of the transformation is referred to as a “transgenote”. The terms “growing”, “producing”, or “regeneration”, as used herein, refers to growing a whole plant from a plant cell, a group of plant cells, a plant part (including seeds), or a plant tissue (e.g., from a protoplast, callus, or tissue part).

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxin and cytokinins. It is sometimes advantageous to add glutamic acid and proline to the medium, especially for such species as corn and alfalfa. Efficient regeneration will depend on the medium, on the genotype, and on the history of the culture. If these variables are controlled, regeneration is reproducible.

Regeneration also occurs from plant callus, explants, organs or parts. Transformation can be performed in the context of organ or plant part regeneration. (see Methods in Enzymology, Vol. 118 and Klee et al., Ann. Rev. Plant Phys. 38:467, 1987). Utilizing the leaf disk-transformation-regeneration method of Horsch et al., Science 227:1229, 1985, disks are cultured on selective media, followed by shoot formation in about 2-4 weeks. Shoots that develop are excised from calli and transplanted to appropriate root-inducing selective medium. Appropriate selection media are known in the art and described by Curry and Cassells in: Plant Cell Culture Protocols, pp. 31-43, Humana Press, Totowa, N.J., 1999; Blackwell et al., IBID 19-30, 1999; Franklin and Dixon in: Plant Cell Culture, pp. 1-25, IRL Press, Oxford, 1994. Rooted plantlets are transplanted to soil as soon as possible after roots appear. The plantlets can be repotted as required, until reaching maturity.

In vegetatively propagated crops, the mature transgenic plants are propagated by the taking of cuttings or by tissue culture techniques to produce multiple identical plants. Selection of desirable transgenotes is made and new varieties are obtained and propagated vegetatively for commercial use. In seed propagated crops, the mature transgenic plants can be self crossed to produce a homozygous inbred plant. The inbred plant produces seed containing the newly introduced heterologous nucleic acid sequences(s). These seeds can be grown to produce plants that would produce the selected phenotype, e.g. pathogen resistance.

Parts obtained from the regenerated plant, such as flowers, seeds, leaves, branches, fruit, and the like are included in the invention, provided that these parts comprise cells that have been transformed as described. Progeny and variants, and mutants of the regenerated plants are also included within the scope of the present invention, provided that these parts comprise the introduced heterologous nucleic acid sequences.

It is contemplated in certain embodiments that a plant cell will be transformed with a recombinant DNA molecule containing at least two DNA sequences or be transformed with more than one recombinant DNA molecule. The DNA sequences or recombinant DNA molecules in such embodiments may be physically linked, by being in the same vector, or physically separate on different vectors. A cell may be simultaneously transformed with more than one vector provided that each vector has a unique selection marker gene. Alternatively, a cell may be transformed with more than one vector sequentially allowing an intermediate regeneration step after transformation with the first vector. Further, it is possible to perform a sexual cross between individual plants or plant lines containing different DNA sequences or recombinant DNA molecules preferably the DNA sequences or the recombinant molecules are linked or located on the same chromosome, and then selecting from the progeny of the cross, plants containing both DNA sequences or recombinant DNA molecules.

Expression of recombinant DNA molecules containing the DNA sequences and promoters described herein in transformed plant cells may be monitored using Northern blot techniques, Southern blot techniques, and/or marker expression, as well as other techniques known to those of skill in the art.

C. Generation of Plants and Portions of Plants with Desirable Traits

The present invention is directed to the surprising discovery that heterologous apoptotic pathway proteins function in plants. Accordingly, such a discovery offers a variety of advantages, including increased resistance of plants and portions of plants (e.g., cut flowers, vegetables, etc.) to biotic and abiotic insults as well as inhibition of senescence. Further, the present invention provides methods of identifying functional plant apoptotic protein homologs and methods of identifying inhibitors and enhancers of apoptotic pathway proteins within the context of a plant cell.

I . Biotic Insult/Agent Resistance

Biotic insults are insults incurred by a plant as the direct or indirect result of a challenge by a biotic agent. Biotic agents include, for example, insects, fungi, bacteria, viruses, nematodes, viroids, mycloplasmas, etc. Biotic agents typically induce programmed cell death in affected plant cells. Such programmed cell death is thought to occur in order to inhibit the spread of an invading pathogen. However, in one embodiment of the present invention, nucleic acid sequences capable of encoding proteins involved in down regulating apoptosis in other organisms are delivered to plant cells and the plants that develop therefrom have a demonstrated resistance to a variety of biotic agents.

In certain embodiments, biotic insult resistant plants offer significant advantages related to crop yield and continued health of ornamentals. Leading biotic agents include various pathogens such as fungi and viruses. An exemplary pathogen, is the fungal pathogen Sclerotinia sclerotiorum, which is one of the most nonspecific and omnivorous plant pathogens known. (see, e.g., Purdy, Phytopathology 69:875-880, 1979). Further, a variety of other economically important pathogens are known including the fungi Botrytis cinerea, Magnaportyhe grisea, Phytophthora spp, Cochliobolus spp, Fusarium graminearum, and Fusarium spp, the Nemtodes Meloidogyne spp (Root knot nematodes), viruses such as tobacco mosaic virus (TMV) and tomato spotted wilt virus (TSWV), tobacco etch virus (TEV), tobacco necrosis virus (TNV), wheat streak mosaic virus (WSMV), soil borne wheat mosaic virus (SBWMV), barley yellow dwarf virus (BYDV), the bacteria Pseudomonas spp and Xanthomonas ssp., as well as many others. A number of examples are available and can be readily identified by those of skill in the art, see, e.g., Plant Pathology, 4^{th} ed., Agrios, ed., Academic Press, San Diego, 1997.

In one embodiment, the present invention provides biotic insult resistant transgenic plants. In this embodiment, heterologous nucleic acid sequences encoding apoptotic pathway proteins are transferred into plants by the methods elucidated above. In certain embodiments the encoded apoptotic pathway protein is an anti-apoptotic protein—that is, the protein negatively regulates either directly or indirectly, the entry of the cell into or the progression of the programmed cell death cycle. Preferred inhibitors include, but are not limited to dominant negative regulators, such as active site cysteine mutated caspase molecules, CARD domains, truncated forms of Apaf-1, etc. as well as other negative regulators such as, IAP, Bcl-2, Bcl-X_{L}, Bcl-W, McL-1, A1, NR13, Ced-9, E1B 19K, BHRF1, and the like. Accordingly, inhibitors exemplified herein include Bcl-2 and Ced-9. These apoptotic inhibitors confer biotic insult resistance upon the plants into which they are transferred. Exemplified biotic insult resistance is pathogen resistance which includes resistance to various viruses and fungi such as tobacco mosaic virus (TMV), tobacco necrosis virus (TNV), tobacco etch virus (TEV), tomato spotted wilt virus (TSWV), Sclerotinia sclerotiorum 1980 (Dickman and Mitra, Physiol. Mol. Plant Pathol. 41:255-263, 1992); Botrylis cinerea (ATCC), Cercospora nicotianae, and Glomerella cingulata. It should be noted, however, that the invention is not limited to resistance to these exemplified pathogens. Accordingly, in certain aspects, expression and application of insecticides and treatment with herbicides may induce a disease state in a plant that leads to an apoptotic event, thus expression of an anti-apoptotic polypeptide may increase the ability of a particular plant to express an insecticidal compound and/or increase the resistance of the particular plant to treatment with various herbicides, thereby resisting an abiotic insult.

2. A Biotic Insult/Agent Resistance

As indicated above, the present invention, also provides increased resistance to abiotic insults. Abiotic agents which cause abiotic insults include, for example, environmental factors such as low moisture (drought), high moisture (flooding), nutrient deficiency, radiation levels, air pollution (ozone, acid rain, sulfur dioxide, etc.), temperature (hot and cold extremes), and soil toxicity, as well as herbicide damage, pesticide damage, or other agricultural practices (e.g., over-fertilization, improper use of chemical sprays, etc.). Accordingly, given that such abiotic agents play an ever increasing role in the viability of a variety of plant types including, food crops and ornamentals, the present invention can be utilized to produce plants with increased resistance to these insults.

In one embodiment, as noted above with regard to biotic insults, the present invention provides abiotic insult resistant transgenic plants. In this embodiment, heterologous nucleic acid sequences encoding apoptotic pathway proteins are transferred into plants by the methods detailed herein. As with biotic insult resistance, these apoptotic inhibitors confer abiotic insult resistance upon the plants into which they are transferred.

One skilled in the art will readily recognize that given the disclosure provided herein, resistance to a particular biotic or abiotic insult/agent can be easily tested using whole plant or leaf sections as appropriate for the method of action of the particular agent.

3. Senescence

Senescence in plants is known to be a regulated process ultimately resulting in cell death (see generally, Guiamet et al., Plant Cell Phys. 31:1123-1130, 1990 for a detailed discussion thereon). Further, it is accompanied by many of the biochemical and structural changes such as induction of cysteine proteases, RNases, etc. that are consistent with programmed cell death. Accordingly, by utilizing the transfer of heterologous apoptotic pathway protein encoding nucleic acid molecules, the present invention provides methods of modulating senescence in plants. While having general application in increase the longevity of a plant, other advantages can be realized. For example, inhibiting senescence can lead to longer shelf-lives for vegetables and fruits, as well as leading to increase longevity and aesthetic appeal of cut flowers and other ornamentals. In addition, in the living plant increased flowering duration and fruit production may be achieved. Accordingly, the present invention has wide utility in both the food stuff market as well as the ornamental market.

In addition, the methods of the present invention may be used to identify endogenous plant programmed cell death modulators which in turn may be selectively over-expressed in the appropriate plant cell to achieve decreased senescence.

D. Methods Utilizing Trans-Species Transfer of Apoptotic Pathway Genes

1. Inhibitors and Enhancers of Apoptotic Activity

Investigation of inhibitors and enhancers of plant apoptosis can provide useful data in the context of designing transgenic plants as well as herbicides and pesticides. Further, with the unexpected and surprising discovery that genes encoding apoptotic pathway proteins from highly diverse species function similarly in plants, plant cells can be used in assays to probe the functionality of transferred apoptotic pathway protein encoding genes. For example, mammalian apoptotic pathway genes such as caspases and Bcl-2 family members may be transferred into plant cells and inhibition or enhancement of apoptosis studied by a variety of methods such as analysis of nucleic acid extracts for identification of apoptotic markers such as DNA ladders, TUNEL staining analysis, and/or microscopy of the cell to identify apoptotic nuclear condensation.

Candidate inhibitors and enhancers may be isolated or procured from a variety of sources, such as human, rodent, fly (D. melanogaster), worm (C. elegans), virus (e.g, vaculoviruses, herpes virus), bacteria, fungi, plants, parasites, libraries of chemicals, peptides or peptide derivatives and the like. Inhibitors and enhancers may be also be rationally designed, based on the protein structure determined from X-ray crystallography (see, Mittl et al., J. Biol. Chem., 272:6539-6547, 1997). In certain preferred embodiments, the inhibitor targets a specific apoptotic pathway protein (e.g., bcl-2 or a homolog thereof).

Without being held to a particular mechanism, the inhibitor may act by preventing processing of an apoptotic pathway zymogen (e.g., a caspase), by preventing enzymatic activity, by preventing interaction of pathway components (either directly or indirectly), or by another mechanism. The inhibitor, itself, may act directly or indirectly. In preferred embodiments, inhibitors of apoptosis in plants decrease programmed cell death as measured by DNA laddering, terminal deoxytransferase-mediated dUTP nick end labeling (TUNEL) (commercial kits available), the level of Ca²⁺ dependent nucleases (Mittler et al., Plant Cell 7:1951-1962, 1995), use of vital stains such as Trypan blue, or similar methods. In other preferred embodiments, the inhibitors are small molecules. In a most preferred embodiment, the inhibitors prevent apoptosis. Inhibitors that can penetrate cells are preferred.

In addition, enhancers of apoptotic activity or apoptotic pathway protein expression are desirable in certain circumstances. At times, increasing apoptosis will mitigate pathogen damage, especially with regard to pathogens that require a living cell to function (e.g., viral or fungal pathogens). Accordingly, pathogen inducible expression of such enhancers can be beneficial. Enhancers may increase the rate or efficiency of apoptotic progression as demonstrated by increased levels of DNA laddering, TUNEL positive cells, up-regulation of Ca²⁺ dependent nucleases (Mittler et al., Plant Cell 7:1951- 1962, 1995), zymogen (caspase or homologs thereof) processing, increase transcription or translation, or act through other mechanisms. As is apparent to one skilled in the art, many of the guidelines presented above apply to the design of enhancers as well.

Screening assays for inhibitors and enhancers will vary according to the type of inhibitor or enhancer and the nature of the activity that is being affected. Assays may be performed in vitro or in vivo. In general, in vitro assays are designed to evaluate apoptotic phenotypic events, DNA laddering, TUNEL staining, Ca²⁺ dependent nuclease levels (Mittler et al., Plant Cell 7:1951-1962, 1995), protein processing or enzymatic activity, and in vivo assays are designed to evaluate such things as reactivity to biotic and abiotic challenges. In any of the assays, a statistically significant increase or decrease compared to a proper control is indicative of resistance, enhancement, or inhibition.

2. Identification of Plant Apoptotic Proteins and Associated Genes

One in vitro assay to investigate apoptotic pathway proteins in plants can be performed by examining the binding of plant cell proteins to mammalian proteins (or other known apoptotic pathway proteins). Briefly, an expression library of plant cDNA can be screened for the ability of the expressed product to bind various mammalian apoptotic pathway components. Similarly, plant extracts can be screened. For example, Apaf-1 protein can be immobilized on a solid surface, such as a column and contacted with expression products of the plant cDNA library. Proteins that bind to mammalian Apaf-1 and are subsequently eluted by a pH gradient, salt gradient, or the like may indicate a plant caspase-9 homolog. Accordingly, any apoptotic pathway protein could be immobilized in a like manner and used to probe a plant cDNA expression library. As one of ordinary skill in the art will appreciate the surprising finding that apoptotic proteins from diverse species can retain functional activity in plants, indicates that any one of the known methods of probing protein-protein interactions may be useful in identifying plant protein homologs for other known apoptotic proteins. One skilled in the art will recognize that a variety of methods of labeling and detection may be used. Such methods include enzyme-linked immunosorbant assays, analytical ultracentrifuge, and use of the BiaCore 3000™ (BiaCore, Uppsula, Sweden).

Accordingly, in one method for identifying plant apoptotic pathway proteins, a fusion protein is constructed comprising a known heterologous apoptotic pathway protein or fragment thereof and a tag peptide sequence (e.g., glutathione-S-transferase) that is bound by an antibody or other molecule (e.g., glutathione). A vector encoding the fusion protein is transformed into bacteria. The fusion protein is purified. In brief, a GST (glutathione-S-transferase) Bcl-2 fusion protein in pGEX-4T-3 (Pharmacia, Uppsala, Sweden) is induced by IPTG and purified using glutathione-beads (see, Kaelin et al., Cell 64:521, 1991). Cells harboring expression library constructs derived from plants may be metabolically labeled. Extracts of the cells are incubated with GST-Bcl-2 charged glutathione-Sepharose beads. Alternatively, the fusion protein may be immunoprecipitated or the like. Unbound protein is washed away, and bound protein is eluted. The bound proteins may be further fractionated by gel electrophoresis, for example. The bound proteins may then be used for raising antibodies, amino acid sequence analysis, and other in vitro tests. Clones encoding the bound proteins may be isolated by any one of a variety of standard methods, including immunoscreening of an expression library, probe hybridization where the probe is based on a partial amino acid sequence, and other known methods.

Once a protein-protein interaction is detected between the plant expression library or plant extract and a known apoptotic pathway component, the protein from the plant expression library can be identified by sequencing and the appropriate cDNA identified.

In one embodiment, methods for detecting plant apoptotic pathway proteins are provided, comprising transfecting or transforming cells with a plant derived expression cDNA library; and detecting interactions between the expressed proteins of the cell and heterologous apoptotic pathway proteins. In another embodiment, methods for detecting putative plant apoptotic pathway proteins are provided, comprising contacting plant extracts or plant proteins derived from an expression library with a known heterologous apoptotic pathway protein and detecting interaction between the heterologous apoptotic pathway protein and the plant derived proteins. In various alternative methods genes from plants or animals can be tested for functional equivalence or screened for apopototic activity by expressing the plant derived gene in an animal cell and the animal derived gene in the plant cell. For example, a cDNA library from a tissue or cell source is constructed in a vector such that a fusion protein is generated with a reporter protein or peptide tag. The reporter or tag can be any protein that allows convenient and sensitive measurement. For example, β-galactosidase and FLAG peptide may be used. Furthermore, multiple tags may be used to allow detection by a sandwich assay. In general, the vector will use a strong promoter to drive expression of the cDNA-fusion genes and an appropriate origin of replication for the host cell. The library of plant genes is then transformed or transfected into animal host cells. Alternatively, animal genes can be tested for functional apoptotic equivalence by transforming a variety of plant host cells (e.g., tobacco-(BY-2) Nagata et al., Int. Rev. Cytol. 132:1-30, 1992; maize (Hi-II), The Maize Handbook, pp. 663-671, Freeling et al. eds., Springer, N.Y., 1994; Arabidopsis (landsberg erecta) Fuerst et al., Plant Physiol. 112:1023-1028, 1996) by any of the methods described herein or other known methods.

Alternatively, additional plant apoptotic pathway proteins may be identified by other methods, such as a yeast two-hybrid system. Briefly, in a two-hybrid system, a fusion of a DNA-binding domain-apoptotic pathway protein (e.g., GAL4-Bcl-2 fusion) is constructed and transformed or transfected into a cell containing a GAL4 binding site linked to a selectable marker gene. A library of cDNAs derived from plant cells and tissues is fused to the GAL4 activation domain is also constructed and co-transformed or co-transfected. When the cDNA in the cDNA-GAL4 activation domain fusion encodes a protein that interacts with Bcl-2, the selectable marker is expressed. Cells containing the cDNA are then grown, the construct isolated and characterized.

Additional assays may be conducted to determine if a protein encoded by a polynucleotide is functionally equivalent to a known apoptosis pathway protein encoded by another polynucleotide, or further, to determine if a plant gene or cDNA has pro- or anti-apoptotic activity. In one embodiments such assays are carried out by expressing the gene of interest heterologously in either of several systems, including, for example, mammalian systems, including human, cells or cell lines in tissue culture, a C. elegans nematode system wherein the nematodes have mutations in the genes ced 9, ced 4, or ced 3 that function in programmed cell death (Accordingly, can look for function restoration.), and the fruitfly, D. melanogaster, in particular under control of an eye-specific promoter in a strain of flies genetically engineered to have small eyes due to excessive apoptosis in the fly retina due to heterologous overexpression or deletion of genes that control apoptosis in the fly eye.

When utilizing mammalian assay systems, expression is by transfection of the gene or cDNA under control of a promoter (e.g, CMV). Cells expressing the gene(s) are observed for evidence of spontaneous induction of apoptosis or are subjected to stimuli that induce apoptosis and the effect of the gene(s) expression on either the kinetics or extent of apoptosis is measured. Apoptosis-inducing stimuli are known to those skilled in the art and include, growth factor withdrawal, ionizing radiation, stimulation with ligands that bind death receptors of the TNF Receptor family (e.g., Fas, TNFα, TRAIL), and heterologous expression of pro-apoptotic genes (e.g., Bax, Bad). Apoptosis in the transfected cells in response to apoptotic stimuli is measured using a number of assays, all known to those skilled in the art, including induction of caspase enzymatic activity (measured by determining the ability of a detergent extract of the cells to cleave the caspase substrate Acetyl-Asp-Glu-Val-Asp-aminomethylcoumarin), DNA degradation (as determined by agarose gel electrophoresis), nuclear condensation (as determined by Hoechst or DAPI staining), cytochrome c release from mitochondria to cytoplasm (measured, for example, either by cell fractionation studies or by immunocytochemistry localization with a cytochrome c monoclonal antibody), and mitochondrial function (measured by reduction of the tetrazoleum salts, MTT or MTS). Accordingly, a gene of interest is determined to be functionally equivalent to a known gene (e.g., Bcl-2, Bax) if its effects on apoptosis (increasing or decreasing either the kinetics or extent of the apoptotic phenotype or inducing apoptosis on its own) are qualitatively and/or quantitatively similar; and in the case of a plant gene, to be a pro- or anti-apoptosis gene if expression of that gene influences the kinetics or extent of apoptosis phenotype or induces apoptosis on its own.

With respect to C. elegans systems, expression of the gene in a ced 9 loss of function mutant worm, in which many or most of the worm cells undergo programmed cell death, would rescue some or all of the cells if the gene encoded an anti-apoptotic protein (e.g., ced 9 rescues in a ced 9 loss of function worm). Expression of the gene in a ced 3 or ced 4 loss of function mutant worm strain in which the 128 cells programmed to undergo apoptosis in the developing hermaphrodite are prevented, would restore the missing cell deaths if the gene was pro-apoptotic.

Further with respect to D. melanogaster systems, the gene would be considered to encode an anti-apoptotic protein if it restored the fly eyes to a normal size and would be considered pro-apoptotic if expression resulted in a further decrease in the size of the fly eye.

In vivo assays are typically performed in cells transformed or transfected either transiently or stably with an expression vector containing an apoptotic pathway protein encoding gene, such as those described herein. In the context of a plant cell in vivo assays will typically include challenge of the cells, plant, or plant portion with a biotic or abiotic agent and the morphology of the inoculation site observed for signs of apoptosis. These inoculation sites can be further characterized by the subsequent analysis for DNA fragmentation and the change of the number of TUNEL positive cells compared to control samples. Further, within the animal cell context such cells may be used to measure caspase processing, substrate turnover, or apoptosis in the presence or absence of a candidate compound. When assaying apoptosis, a variety of cell analyses may be used including, for example, dye staining and microscopy to examine nucleic acid fragmentation and porosity of the cells. Further, in vivo assaying for the ability of the transformed or transfected apoptotic pathway protein or protein activated thereby to cleave known substrates that are co-transfected, co-transformed, or placed in the cell culture media in the presence of the candidate compound can be performed thereby allowing for the detection and determination of substrate turnover.

Detection methodologies for apoptotic pathway enzymes are those commonly used to analyze enzymatic reactions and include, for example, SDS-PAGE, spectroscopy, HPLC analysis, autoradiography, chemiluminescence, chromogenic reactions, and immunochemistry (e.g., blotting, precipitating, etc.). Further, a variety of detection methods for observing programmed cell death (i.e., apoptosis) in plants are available. See for example, Mittler et al., Plant Cell 7:29-42, 1995; Mittler et al., Plant Mol. Biol. 34:209-221, 1997; TUNEL kits from Oncor and Boehringer-Mannheim.

When analysis of caspase activity is desired, for example, when putative plant apoptotic pathway genes are expressed in an animal cell or where otherwise necessary, chromogenic substrates are preferably utilized. The turnover of these substrates measures, directly or indirectly, the apoptotic pathway and, in particular, the enzymatic activity of one or more caspase molecules. In this regard a variety of substrates such as labeled caspase molecules, lamin, PARP and caspase substrate analogues are known by those of skill in the art. Such substrates are also available commercially from such companies as Oncogene Research Products, Cambridge, Mass. Illustrative substrate analogues which are tagged with fluorescent markers include, ZEVD-amc (carbobenzoxy-Glu-Val-Asp-aminomethylcoumarin), YVAD-amc (Acetyl-Tyr-Val-Ala-Asp-aminomethylcoumarin), and DEVD-amc (Acetyl-Asp-Glu-Val-Asp-aminomethylcoumarin).

Furthermore, in other assay embodiments, eukaryotic promoters may be utilized within a construct for delivering either inducible or constitutively expressed pro- or anti-apoptotic proteins to cells for assaying. For example, cells can be transformed or transfected such that they overexpress the anti-apoptotic polypeptide Bcl-2, thereby providing cells wherein membrane preparations would have a higher level of Bcl-2, such that only enhancers of apoptosis which were capable of overcoming Bcl-2 inhibition would be detected. Accordingly, the cell could be transformed with a second vector containing an inducible cDNA from a plant expression library. In this regard, such cells could be treated with a stimulus of apoptosis such that the cell is “poised” for apoptosis prior to induction of the cDNA transcription.

The methods described above for identification of inhibitors and enhancers of apoptosis provides an alternative format for measuring apoptotic activity, in that a cell is treated so that it is “poised” for programmed cell death. In this way the cell has synthesized and/or activated all necessary components that are required for programmed cell death. All that is required is a stimulus to cause the cell to extend past its holding point and into apoptosis. Accordingly, an enhancer would cause the cell to progress into programmed cell death, while an inhibitor would delay or suppress this progress in the presence of an apoptotic stimulus (e.g., anti-fas antibodies, staurosporine, and the like).

The holding point which prevents the cell from proceeding into programmed cell death can be the overexpression of a apoptotic pathway protein that is a cell survival (anti-apoptotic) polypeptide or treatment of the cells with known apoptotic inhibitors. Anti-Apoptotic polypeptides are characterized in that they exhibit the ability to prevent apoptosis when expressed or activated in a cell induced to undergo apoptosis. For example, in the absence of a functioning anti-apoptotic polypeptide, a cell treated with an apoptotic enhancer (e.g., a pro-apoptotic agent) will initiate or accelerate apoptosis. However, in the presence of a an apoptotic polypeptide, treatment with a pro-apoptotic agent/enhancer can initiate the programmed cell death pathway, but the cell will survive due to inhibition of one or more events along the pathway. Depending upon the point at which the anti-apoptotic polypeptide functions, the programmed cell death pathway can be inhibited early or relatively late within the execution of the cascade of events leading to ultimate cell death. Anti-apoptotic polypeptides and their encoding nucleic acids are well known in the art and include, for example, the Bcl-2 family of related proteins Bcl-2, Bcl-X_{L}, Mcl-1, E1B-19K, IAP, Ced-9, Bcl-w, etc., as well as the dominant-negative forms of the caspases. These forms include, for example, caspase's with an inactivating mutation of the active site cysteine.

Overexpression of a an anti-apoptotic polypeptide can be achieved using, for example, recombinant methods known to those skilled in the art. Routine procedures for performing such recombinant expression methods are described in, for example, Sambrook et al., Molecular Cloning: A Laboratory Manual, Cold Spring Harbor Laboratory, New York (1992), Greene Publishing Associates and Wiley-Interscience, New York, (1995). Such methods can be used to express stably or transiently an anti-apoptotic polypeptide at a level which is sufficient to prevent the induction of apoptosis. The nucleic acid molecule encoding the anti-apoptotic polypeptide can be encoded by, for example, a homologous nucleic acid derived from the same species or cell type, or alternatively, the nucleic acid molecule can be encoded by a heterologous nucleic acid derived from a different species or cell type. The source of the encoding nucleic acid is not important so long as the encoded anti-apoptotic polypeptide exhibits apoptosis inhibiting activity.

A level of expression of an anti-apoptotic polypeptide which is sufficient to prevent the induction of apoptosis is known to those skilled in the art and can also be routinely determined by those skilled in the art. Expression vectors and systems are known and commercially available which provide for recombinant polypeptide expression. It is a routine matter for one skilled in the art to choose a vector or system which will provide sufficient levels of expression in a particular host cell. Alternatively, the expression level sufficient to prevent the induction of apoptosis can be routinely determined by expressing the anti-apoptotic polypeptide and then measuring whether the cell survives after treatment with a pro-apoptotic agent; or induction of the plant derived cDNA molecule.

In addition to recombinant methods of over-expressing an anti-apoptotic polypeptide, a cell can be used which inherently over-expresses an anti-apoptotic polypeptide. A specific example of a cell inherently over-expressing an anti-apoptotic polypeptide is the B cell lymphoma in which Bcl-2 was initially identified. This leukemia has a translocation of chromosome 14 to 18 causing high level expression of Bcl-2 and therefore cell survival. Accordingly, the leukemic phenotype is due to the increased cell survival. Other cell lines which inherently over-express an anti-apoptotic polypeptide can similarly be used in the methods of the invention.

The block from apoptosis due to over-expression of an anti-apoptotic polypeptide and the treatment of the cells with a pro-apoptotic agent provides antagonistic influences to the cell. In this way, the cells are essentially poised for programmed cell death. A pro-apoptotic agent can be a variety of different insults to the cell including, molecular, environmental and physical stimuli. As defined previously, such stimuli are known to those skilled in the art and can be characterized by activating a molecule within the apoptotic pathway. Examples of pro-apoptotic agents include inducers such as deprivation of a growth factor, Fas ligand, anti-Fas antibody, staurosporine, Tumor Necrosis Factor, ultraviolet and gamma-irradiation. Thus, treatment of a cell over-expressing an anti-apoptotic polypeptide with a pro-apoptotic agent will prime the cell for apoptosis since both positive and negative signals provide balancing effects. One advantage of this priming is that all cell death components are available for apoptosis once a signal is received that overcomes the block of the anti-apoptotic polypeptide. This allows for the rapid induction of apoptosis which can be use in screening for compounds that possess apoptosis inducing activity in the presence of Bcl-2 or Bcl-X_{L} or related molecules. Such cells are particularly useful in screening plant derived cDNA expression libraries for pro-apoptotic polypeptides. Similarly, pro-apoptotic polypeptides can be expressed under the direction of an inducible promoter. Accordingly, these cells can contain plant derived cDNA expression constructs under constitutive expression. Thus, if induction does not lead to cell death a cell death regulator derived from the expressed cDNA is putatively present.

An anti-apoptotic protein derived from a plant derived cDNA expression library may also be identified by a method comprising, transforming host cells such as mammalian cells with a plant cDNA expression library, contacting the cells with an apoptotic inducer such as staurosporine, gamma-irradiation, anti-Fas, or an Fas ligand, or the like and detecting cells that do not enter apoptosis, thereby identifying a cell and a cDNA construct expressing an anti-apoptotic protein. The expression construct can then be sequenced and the sequence elucidated.

3. High Throughput

The methods described herein are also amenable to high throughput formats (e.g., a multi-well format assay where large numbers of samples can be screened rapidly and efficiently). For example, a 96-well format provides practical advantages since plates appropriate for manipulations and measuring devices are commercially available. Such procedures can be further automated to increase further the speed and efficiency of the method. These features, combined with the specificity of the method, allow for cell-free high throughput screening of candidate inhibitors or enhancers of apoptotic activity.

E. Pharmaceutical Applications

Inhibitors and enhancers of programmed cell death may be used in the context of this invention to exert control over the cell death process. Thus, these inhibitors and enhancers will have utility in diseases characterized by either excessive or insufficient levels of apoptosis and protection of plants from biotic and abiotic insults. Accordingly, inhibitors of apoptosis in plants may have potential human therapeutic value to treat the major neurodegenerative diseases: stroke, Parkinson's Disease, Alzheimer's Disease, and ALS. As well, inhibitors may be used to inhibit apoptosis in the heart following myocardial infarction, in the kidney following acute ischemia, and in diseases of the liver.

Pharmaceutical compositions also are provided by the present invention. These compositions may contain any of the above described inhibitors, enhancers, DNA molecules, vectors or host cells, along with a pharmaceutically or physiologically acceptable carrier, excipients or diluents. Generally, such carriers should be nontoxic to recipients at the dosages and concentrations employed. Ordinarily, the preparation of such compositions entails combining the therapeutic agent with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents.

In addition, the pharmaceutical compositions of the present invention may be prepared for administration by a variety of different routes, including for example intraarticularly, intracranially, intradermally, intrahepatically, intramuscularly, intraocularly, intraperitoneally, intrathecally, intravenously, subcutaneously or even directly into a tumor. In addition, pharmaceutical compositions of the present invention may be placed within containers, along with packaging material which provides instructions regarding the use of such pharmaceutical compositions. Generally, such instructions will include a tangible expression describing the reagent concentration, as well as within certain embodiments, relative amounts of excipient ingredients or diluents (e.g., water, saline or PBS) which may be necessary to reconstitute the pharmaceutical composition. Pharmaceutical compositions are useful for both diagnostic or therapeutic purposes.

Pharmaceutical compositions of the present invention may be administered in a manner appropriate to the disease to be treated (or prevented). The quantity and frequency of administration will be determined by such factors as the condition of the patient, and the type and severity of the patient's disease. Dosages may be determined most accurately during clinical trials. Patients may be monitored for therapeutic effectiveness by appropriate technology, including signs of clinical exacerbation, imaging and the like.

In other embodiments, plant derived apoptotic pathway proteins may be delivered to cells as part of gene delivery vehicles. In many diseases and syndromes, too little apoptosis is an important feature in their development. Treatment of many autoimmune diseases and tumors would benefit from increased apoptosis. One means to increase apoptosis is to provide target cells with caspase genes in an expressible form. This may be accomplished by delivery of DNA or cDNA capable of in vivo transcription of a pro-apoptotic polypeptide. More specifically, in order to produce an apoptotic polypeptide in vivo, a nucleic acid sequence coding for the apoptotic polypeptide is placed under the control of a eukaryotic promoter (e.g., a pol III promoter, CMV or SV40 promoter). Where it is desired to more specifically control transcription, a apoptotic polypeptide may be placed under the control of a tissue or cell specific promoter (e.g., to target cells in the liver), or an inducible promoter, such as metallothionein.

Many techniques for introduction of nucleic acids into cells are known. Such methods include retroviral vectors and subsequent retrovirus infection, adenoviral or adeno-associated viral vectors and subsequent infection, and complexes of nucleic acid with a condensing agent (e.g., poly-lysine). These complexes or viral vectors may be targeted to particular cell types by way of a ligand incorporated into the vehicle. Many ligands specific for tumor cells and other cells are well known in the art.

A wide variety of vectors may be utilized within the context of the present invention, including for example, plasmids, viruses, retrotransposons and cosmids. Representative examples include adenoviral vectors (e.g., WO 94/26914, WO 93/9191; Yei et al., Gene Therapy 1:192-200, 1994; Kolls et al., PNAS 91(1):215-219, 1994; Kass-Eisler et al., PNAS 90(24):11498-502, 1993; Guzman et al., Circulation 88(6):2838-48, 1993; Guzman et al., Cir. Res. 73(6):1202-1207, 1993; Zabner et al., Cell 75(2):207-216, 1993; Li et al., Hum Gene Ther. 4(4):403-409, 1993; Caillaud et al., Eur. J. Neurosci. 5(10):1287-1291, 1993), adeno-associated type 1 (“AAV-1”) or adeno-associated type 2 (“AAV-2”) vectors (see WO 95/13365; Flotte et al., PNAS 90(22):10613-10617, 1993), hepatitis delta vectors, live, attenuated delta viruses and herpes viral vectors (e.g., U.S. Pat. No. 5,288,641), as well as vectors which are disclosed within U.S. Pat. No. 5,166,320. Other representative vectors include retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218.

Within certain aspects of the invention, nucleic acid molecules that encode the apoptotic pathway protein may be introduced into a host cell utilizing a vehicle, or by various physical methods. Representative examples of such methods include transformation using calcium phosphate precipitation (Dubensky et al., PNAS 81:7529-7533, 1984), direct microinjection of such nucleic acid molecules into intact target cells (Acsadi et al., Nature 352:815-818, 1991), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS 89:6094, 1990), lipofection (Felgner et al., Proc. Natl. Acad. Sci. USA 84:7413-7417, 1989), microprojectile bombardment (Williams et al., PNAS 88:2726-2730, 1991), polycation compounds such as polylysine, receptor specific ligands, liposomes entrapping the nucleic acid molecules, spheroplast fusion whereby E. coli containing the nucleic acid molecules are stripped of their outer cell walls and fused to animal cells using polyethylene glycol, viral transduction, (Cline et al., Pharmac. Ther. 29:69, 1985; and Friedmann et al., Science 244:1275, 1989), and DNA ligand (Wu et al, J. of Biol. Chem. 264:16985-16987, 1989), as well as psoralen inactivated viruses such as Sendai or Adenovirus. Appropriate vehicles also include gene activated matrices as described in U.S. Pat. No. 5,763,416 and WO 97/38729, U.S. Ser. No. 08/631,334.

The following examples are offered by way of illustration, and not by way of limitation.

### EXAMPLES

### Example I

### Construction of an IAP Containing Binary Vector

The open reading frame (ORF) within the vector pOPIAPRC (Birmbaum et al., J. of Virology 68:2521-2528, 1994) was engineered by PCR to introduce an Nco I site at the 5′ end and Bam HI site at the 3′ end. This manipulation introduced an Ala residue between the Met (position 1) and Ser (position 2) residues of the native protein. The PCR conditions used were as follows: 1 min 94° C.; 1 min 45° C.; 2 min 72° C. for 2 cycles followed by 35 additional cycles of 1 min 94° C.; 1 min 55° C.; 2 min 72° C.

Primer prc-5:
5′-gttgcagaccatggccagctcccgagcattggc-3′
Primer pre-3:
5′-ttttggatccttttattgttacacttgg-3′

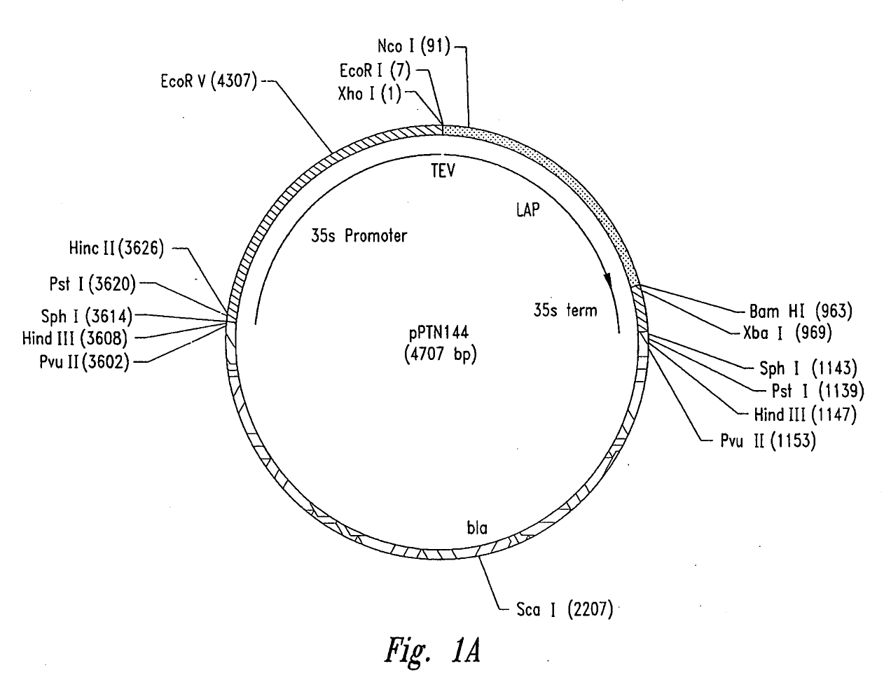

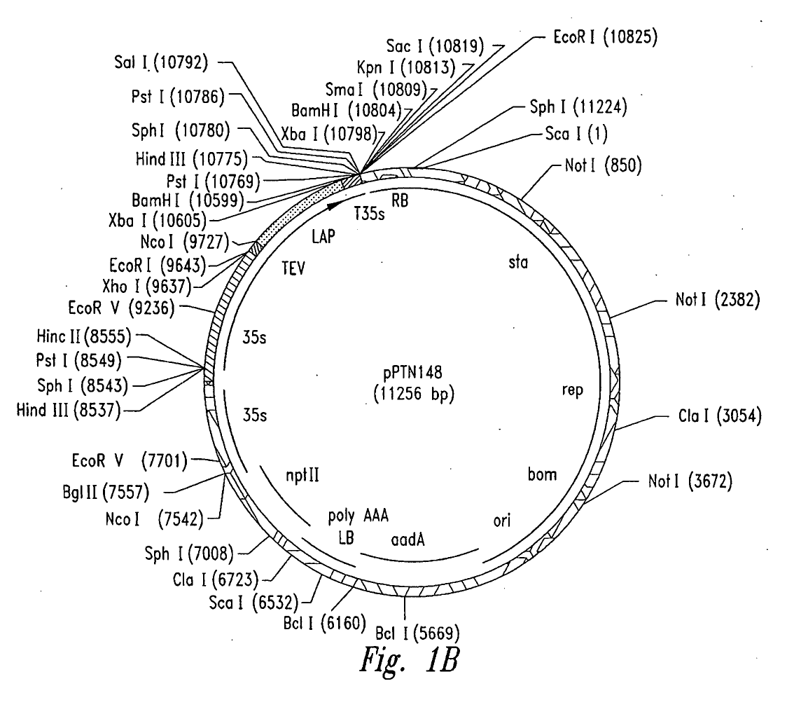

The PCR product was digested with Nco I and Bam HI and subcloned in the plant expression cassette pRTL2 (described by Carrington et al., J. of Virology 64:1590-1597, 1990 as pRTLGUS). The resultant vector is referred to as pPTN144 (FIG. 1A). The 35S-IAP cassette of pPTN144 was subcloned using Hind III into the binary vector pZP212 (Hajdukiewicz et al., Plant Mol. Bio 25:989-994, 1994). The resultant vector is referred to as pPTN148 (FIG. 1B).

### Example II

### Construction of a Ced-9 Containing Binary Vector

The ORF of Ced-9 was engineered by PCR to introduce an Nco I site at the 5′ end and an Xba I site at the 3′ end. This manipulation introduced an Ala residue between the Met (position 1) and Thr (position 2) residues of the native protein. The PCR conditions were identical to those described for the engineering of the pOPIAPRC ORF.

Primer ced-5:
5′-gaattccggtttgagccatggcgacacgctgcacggcg-3′
Primer ced-3-2:
5′-ttttttctagaaatacgttacttcaagctg-3′

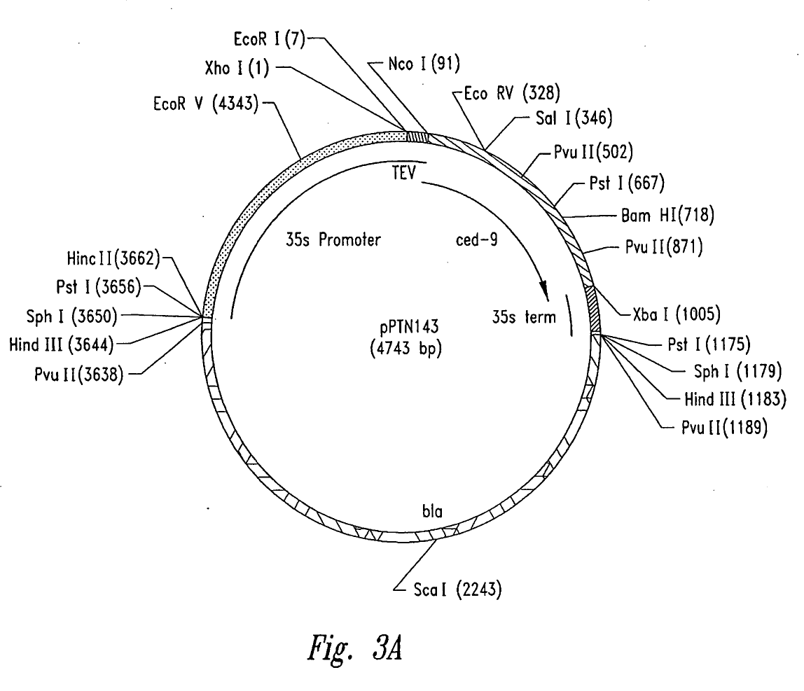

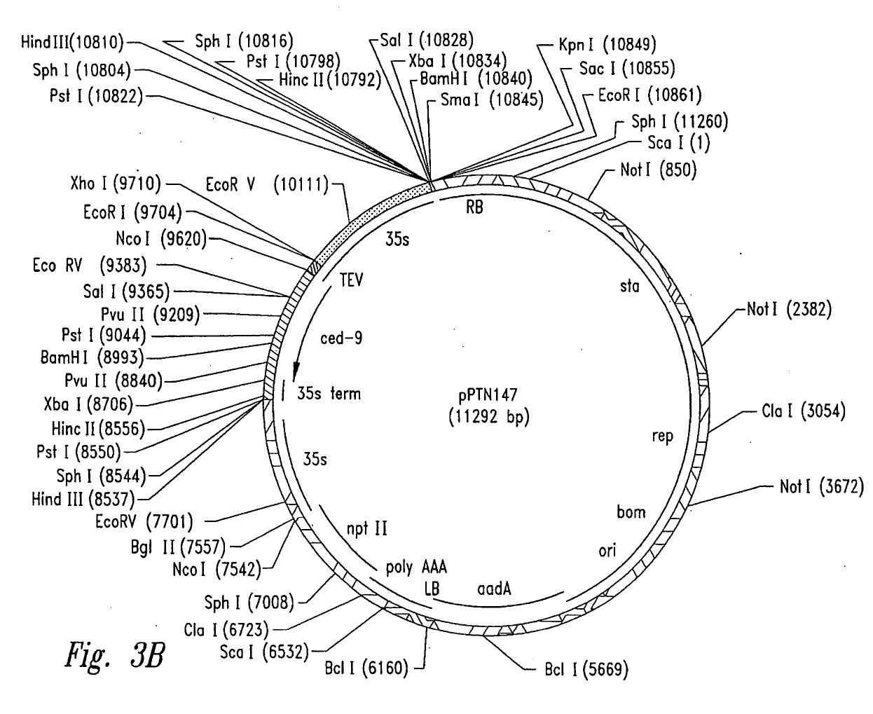

The PCR product was digested with Nco I and Xba I and subcloned into the plant expression cassette pRTL2 (Carrington et al., J. of Virology 64:1590-1597, 1990). The resultant vector is referred to as pPTN143 (FIG. 3A). The ced-9 plant expression cassette from pPTN143 was subcloned as a Hind III fragment into the binary vector pZP212 (Hajdukiewicz et al., Plant Mol. Bio 25:989-994, 1994). The resultant vector is referred to as pPTN 147 (FIG. 3B).

### Example III

### Construction of a Bcl-2 Containing Binary Vector

The cDNA of bcl-2, was engineered to introduce an Nco I site at the 5′ end of the ORF for bcl-2 and an Xba I site at the 3′ end of the ORF. PCR reactions were carried out with the following primers using the identical conditions recited in Example I:

Primer Bcl2-5:
5′-tttttcctctgggagggccatggcgcacgctgg-3′
Primer Bcl2-3:
5′-ttttttctagatgctcttcgggcgtgg-3′

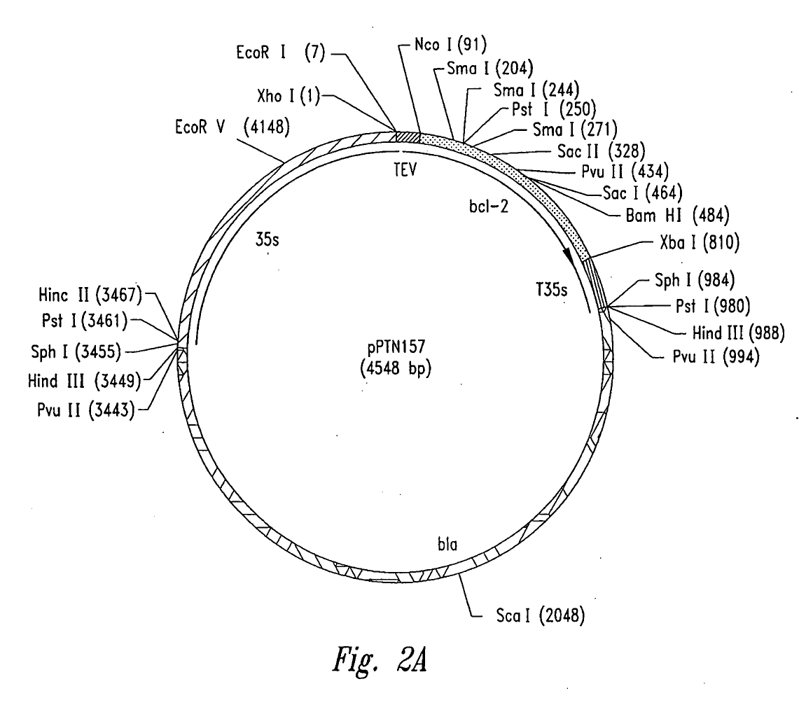

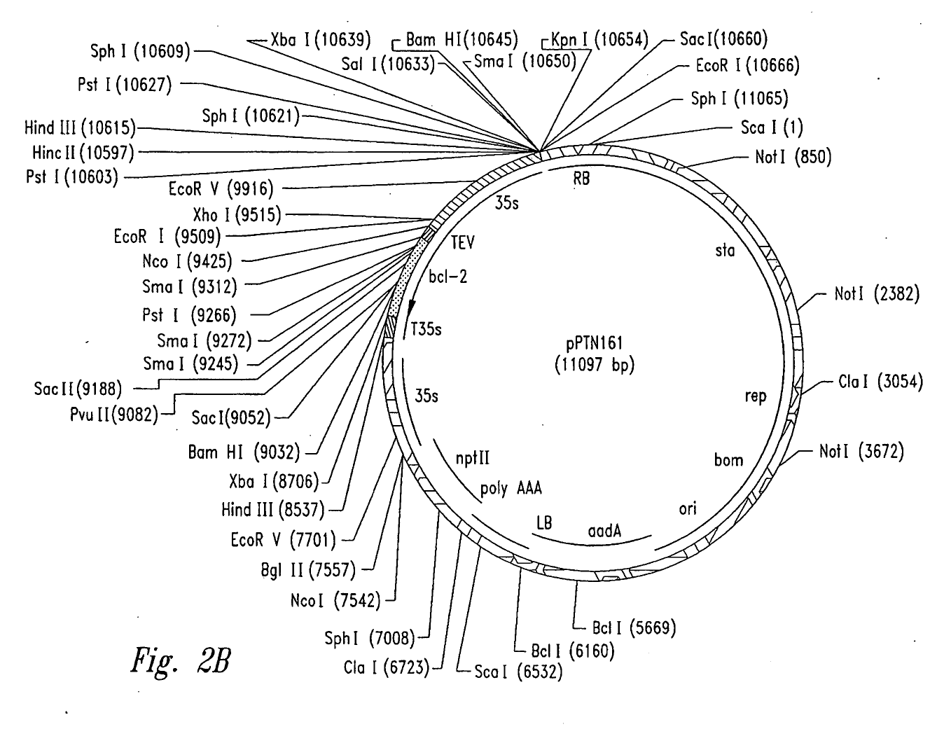

These manipulations introduced an Ala residue between the Met (position 1) and His (position 2) residues of the native protein. The PCR product was then digested with Nco I and Xba I and subcloned into the plant expression cassette pRTL2. The resultant vector is referred to as pPTN157 (FIG. 2A). The full bcl-2 plant expression cassette was then subcloned into the binary vector pZP212 using the endonuclease Hind III followed by ligation. The resultant vector is referred to herein as pPTN161 (FIG. 2B).

### Example IV

### Construction of an E1B 19K Containing Binary Vector

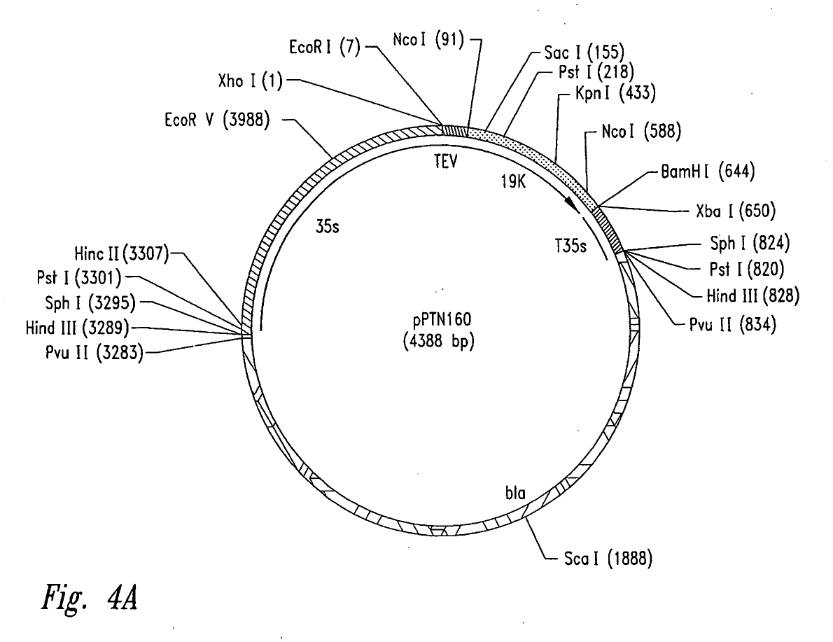

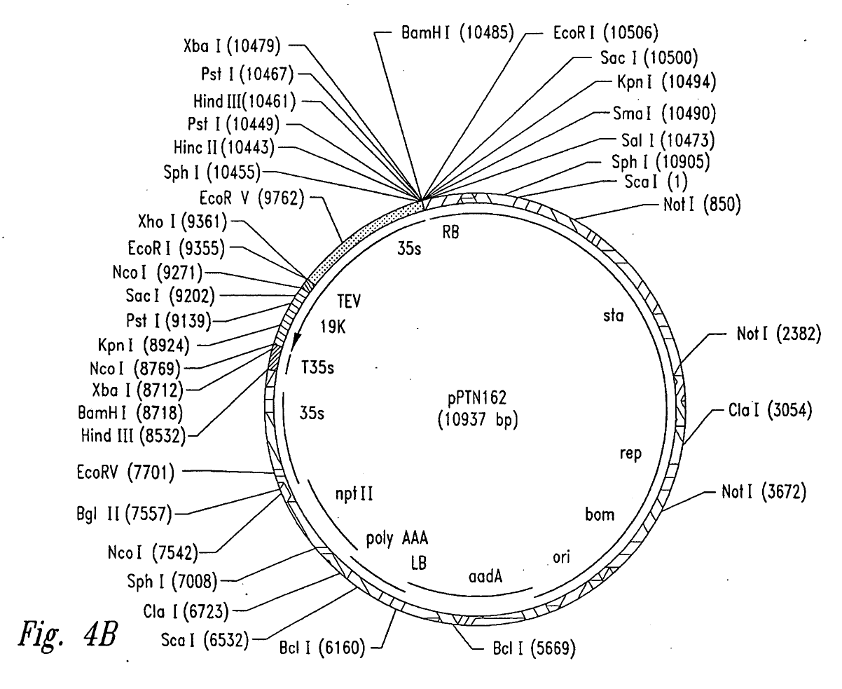

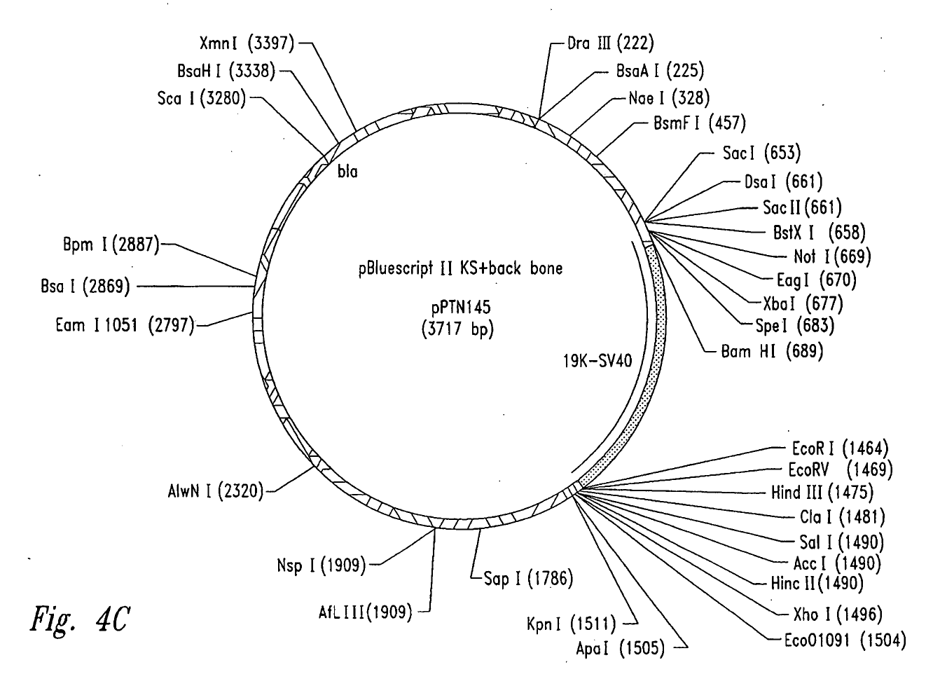

The Eco RI/Bam HI fragment within the vector pCMV19K (White and Cipriani, Mol. Cell. Biol. 10:120-130, 1990) was subcloned into pBluescript KS⁺ for sequence analysis. The resultant vector is referred to as pPTN145 (FIG. 4C). Based on the sequence data obtained from the template pPTN145 primers were designed to introduce an Nco I site at the 5′ end of the ORF and a Bam HI site at the 3′ end of the ORF by PCR. This manipulation did not add an additional residue within the protein.

Primer pPTN 145-5:
5′-gcttcctgaactccatggaggcttggg-3′
Primer pPTN 145-3:
5′-tttttggatccaacattcattcccgaggg-3′

Due to an internal Nco I site within the E1B-19K ORF the fragment from pPTN 145 was subcloned into pRTL2 (NcoI/Bam HI) as a triple ligation NcoI/Kpn I and Kpn I/Bam HI. The resultant vector is referred to as pPTN160 (FIG. 4A). The 35S-19k cassette from pPTN160 was subcloned into the binary vector pZP212 as a Hind III fragment. The resultant vector is referred to as pPTN162 (FIG. 4B).

### Example V

### Plant Transformation Protocol

Agrobacterium Strains:

Agrobacterium tumefaciens strain, C58C1 (Mol. Gen. Genet. 204:383, 1986), was used in all tobacco transformations. The binary vectors pPTN147, pPTN148, pPTN161 and pPTN162 were mobilized into C58C1 by tri-parental mating (Ditta et al., Proc. Natl. Acad. Sci. USA 77:7347-7351, 1980). Agrobacterium transconjugants were selected on AB minimal medium supplemented with 100 mg/L Streptomycin, 100 mg/L Spectinomycin, 50 mg/L Rifampicin, 50 mg/L Gentamycin and 1.5 sucrose.

Wild type tobacco Nicotiana tabacum cultivar Glurk (genotype NN) or N. tabacum cultivar Turkish (genotype nn) were used for pathogen inoculation, and for construction of transgenic plants. (Both Glurk and Turkish cultivars are available from a variety of sources including the University of Nebraska at Lincoln, Collection).

Tobacco transformations were conducted using a modification of the leaf dip protocol (Horsch et al., Science 227:1229-1231, 1985). Explants were prepared from the first two leaves of 30-40 day old plants. Following a 3 day co-cultivation period, explants were subcultured to MS/B5 medium supplemented with 1 mg/l BAP, 0.1 mg/l NAA and 100 mg/l Kanamycin. The antibiotics Carbenicillin and Cefotaxime were added to counter select against Agrobacterium. Explants were subcultured every 2 weeks to fresh medium. Differentiated Kanamycin tolerant shoots were excised after 6-8 weeks in culture. Shoots were rooted on a MS/B5 based medium supplemented with 0.1 mg/l NAA and 50 mg/l Kanamycin.

### Example VI

### Screening Assays for Transgenic Lines

Kanamycin resistant transgenic plants were propagated on Murashige-5k00G basal salts medium (Sigma), supplemented with Kanamycin sulfate (100 mg/l), NAA (auxin), 0.1 mg/l and BAP (cytokinin) 1 mg/l.

For expression studies, tobacco leaves were collected, frozen in liquid nitrogen and ground to a powder. Plant RNA was isolated as described in Example VIII. RNA blots were hybridized with radiolabeled pPN147, pPTN148, pPTN1612 or pPTN162. 18S rRNA served as an internal control to insure equivalent RNA loading. RNA blot hybridization and membrane washing were performed under stringent conditions (Sambrook et al., supra).

Western Analysis:

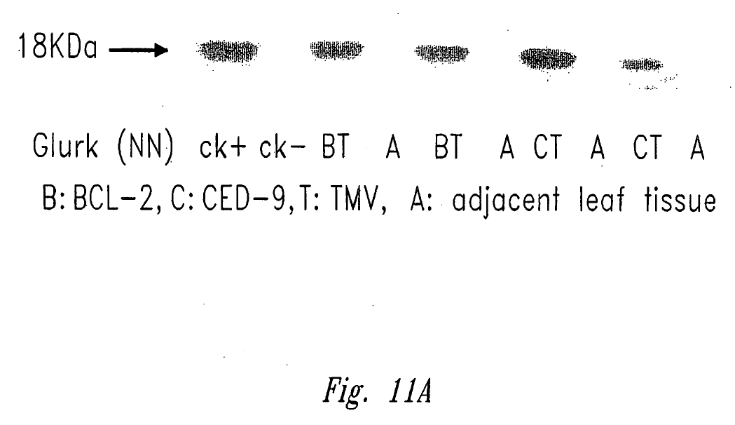

TMV proteins were detected by Coomassie blue stained and/or—immunoassays from SDS PAGE. Infected plant tissue was ground in ddH20 (0.2 g tissue/l ml) and boiled 1-5 min. in five volumes of Laemmli loading buffer (Laemmli, Nature 227:680-685, 1970). Approximately 15 μl were applied to 12% SDS-polyacrylamide gels. Proteins were electrophoretically transferred to nitrocellulose membranes, and then detected with a TMV polyclonal antibody made against virions diluted 1:1000 (available from the ATCC PVAS-135, Manassass, Va.) using colorometric (Immun-Blot kit, BioRad) assays (FIGS. 11A and 11B)

Northern and Southern Analysis:

Southern analysis (data not shown) and Northern blots indicated both the presence and expression of the four transgenes.

### Example VII

### Pathogen Inoculation and Resistance Assays

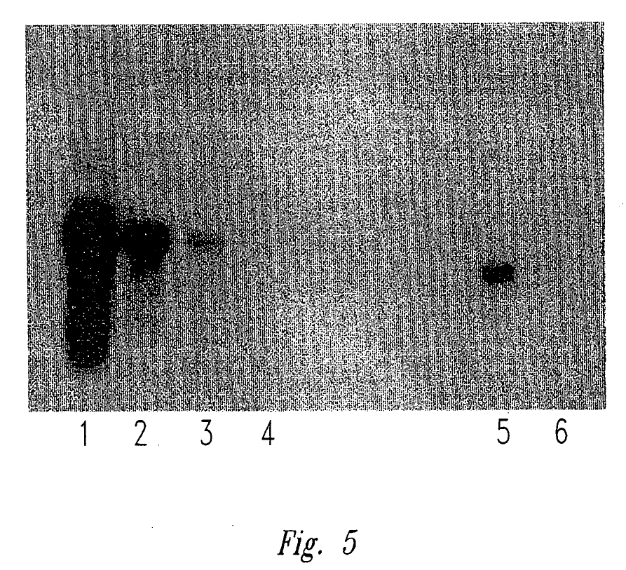

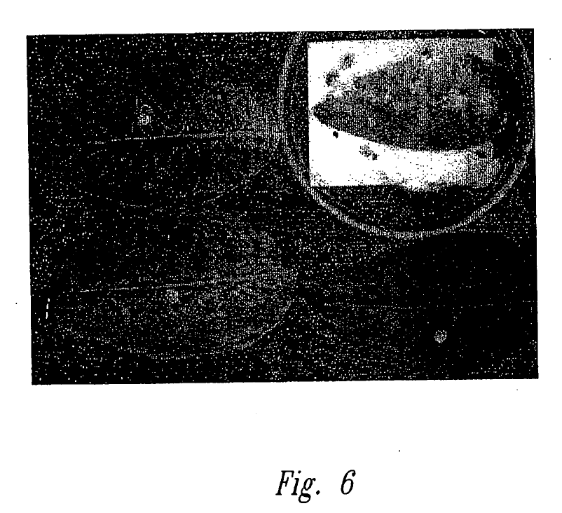

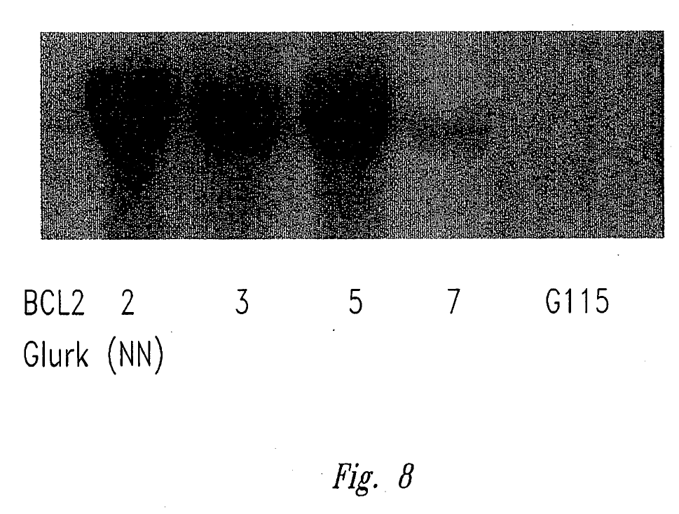

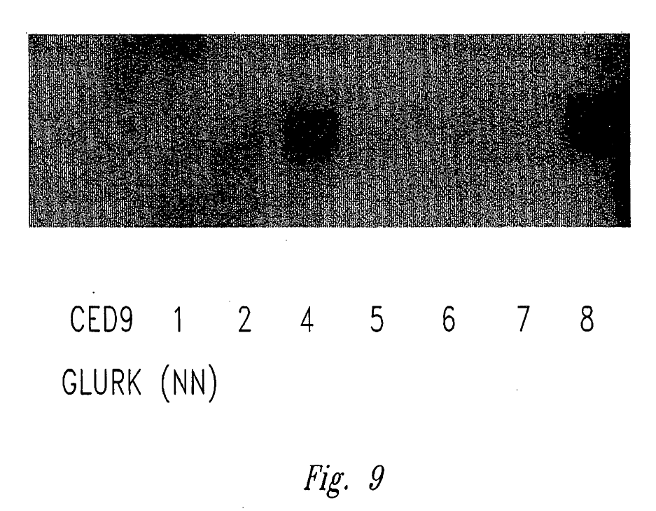

Evaluation of plant resistance to was investigated tobacco mosaic virus (TMV). In this experiment tobacco cultivars with (Glurk) and without (Turkish) the N gene for TMV resistance were transformed. A minimum of 10 kanamycin resistant Glurk and Turkish lines were obtained with each of the four transgenes. Southern analysis (data not shown) and Northern blots indicated both the presence and expression of the four transgenes (FIG. 5, demonstrates IAP and Ced-9 expression, while FIGS. 8 and 9 show Bcl-2 and Ced-9 expression, respectively). There was no correlation between copy number, level of expression and plant response. There were no detectable physiological differences between the transgenic plants when compared with wild type controls. All plants flowered and set seed in a similar manner. Randomly chosen single copy insertion kanamycin resistant tobacco were evaluated for response to pathogen challenge.

Transgenic tobacco were tested for resistance to tobacco mosaic virus (TMV) tobacco necrosis virus (TNV), tobacco etch virus (TEV), tomato spotted wilt virus (TSWV), Sclerotinia sclerotiorum 1980 (Dickman and Mitra, Physiol. Mol. Plant Pathol. 41:255-263, 1992); Botrytis cinerea (ATCC), Cercospora nicotianae, and Glomerella cingulata. Transgenic and control tobacco were grown at 25° C. with 16 h light periods in a greenhouse.

Viral Assays:

Detached tobacco leaves were dusted with carborundum and rub-inoculated with 110 μg TMV in 50 μl H₂O. After 5 min leaves were thoroughly washed with water, placed in petri dishes and held at 25° C. under constant illumination. Inoculations with TNV and TSWV were similar except that infected plant sap diluted 1/25 (w/v) in 10 mM Na phosphate buffer (pH 7) was used for inoculum.

Effects of anti-apoptotic gene expression on TMV in both resistant (NN) and susceptible (nn) tobacco lines was effected. The N gene confers a hypersensitive response to TMV infection which prevents systemic spread of the virus. Necrotic lesions appear 4 to 5 days after inoculation with TMV and continue to expand in size over time. Local lesion development appears to be bi-phasic in nature. Initially small (1-2 mm) water-soaked lesions develop after two to three days which then become uniformly necrotic. After this, the necrosis continues to expand with chlorotic, rather than water-soaked margins. Virus movement is restricted to the vicinity of the lesion which grow and subsequently coalesce, resulting in death of the entire leaf. TMV induces no visible symptoms on inoculated leaves of tobacco without the N gene but does replicate and spread throughout the leaf. TMV was inoculated on the four different transgenic Glurk (NN) lines (using a minimum of three different lines) as well on two Turkish (nn) lines. Untransformed tobacco and tobacco containing vector alone and vector only plants served as controls.

Further, detached leaf assays were used in most experiments as they are experimentally advantageous and demonstrated a 100% correlation in response to pathogen challenge when compared to whole plants in a greenhouse.

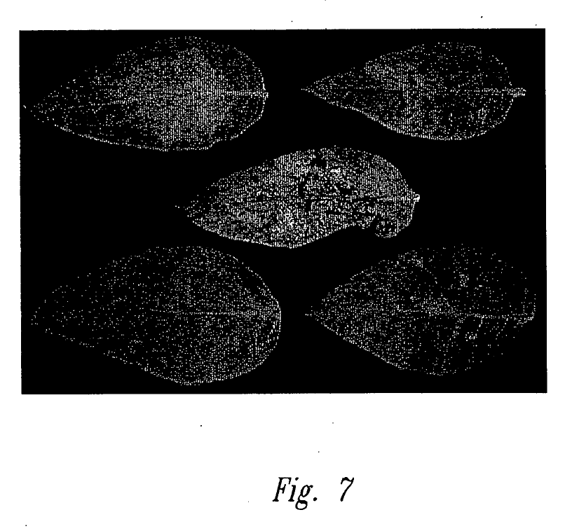

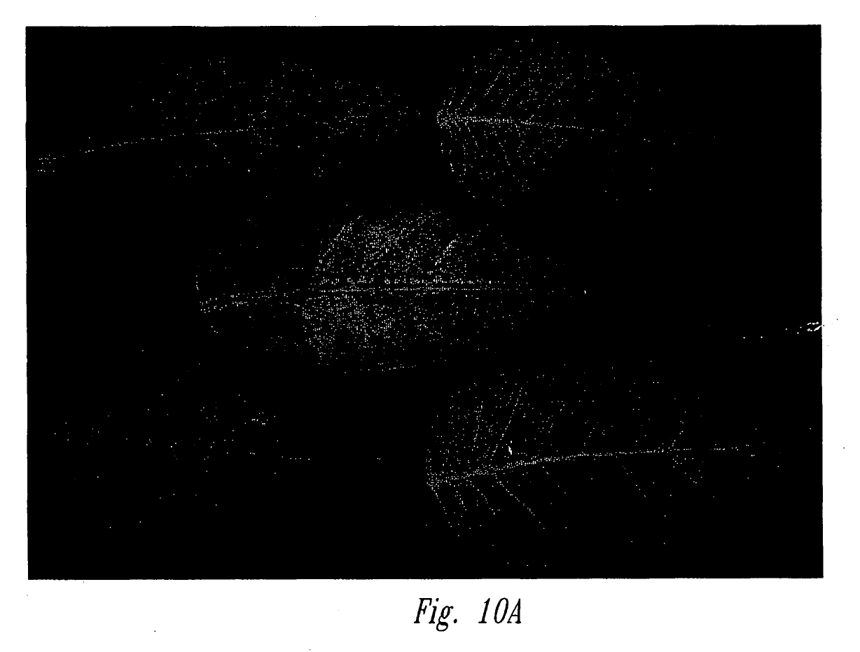

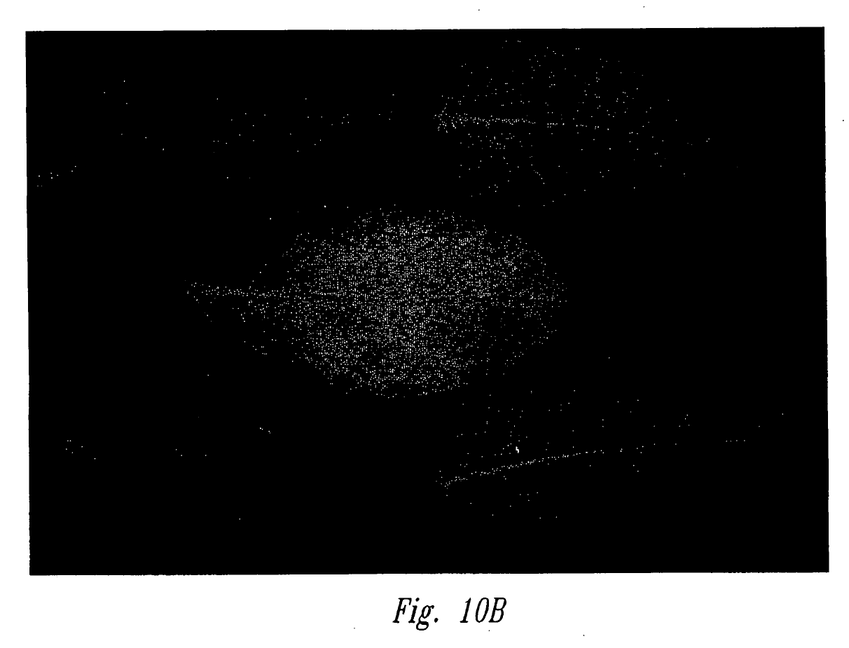

TMV inoculation gave rise to local lesions on Glurk lines both with and without the Bcl-2, CED 9 and IAP genes. In all of these studies, the adenovirus E1B-19k containing transgenic plants in either tobacco cultivars did not appear resistant to this pathogen and thus were not evaluated further. Secondary growth of the lesions was greatly restricted, however, in those lines expressing the other transgenes (FIGS. 7, 10A, and 10B). Thus Bcl-2, CED-9 and IAP appear to potentiate the resistance provided by the N gene by preventing lesion expansion. It was possible that TMV could be spreading out of the initial lesions without inducing further necrosis. To determine this, Western blot analysis was done separately on local lesions and surrounding tissues (FIG. 11A). While virus was abundant within lesions, no virus was detected outside lesion boundaries confirming that N gene resistance was indeed potentiated by Bcl-2 and ced-9. On the other hand, Bcl2 and ced-9 had no effect on TMV accumulation in the Turkish lines (FIG. 11B).

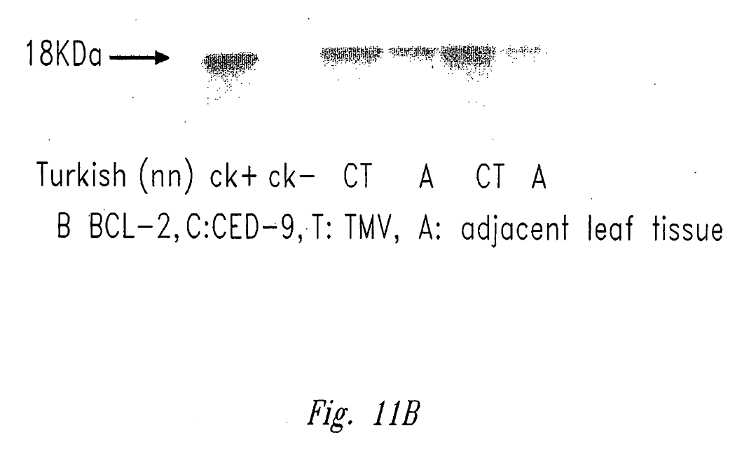

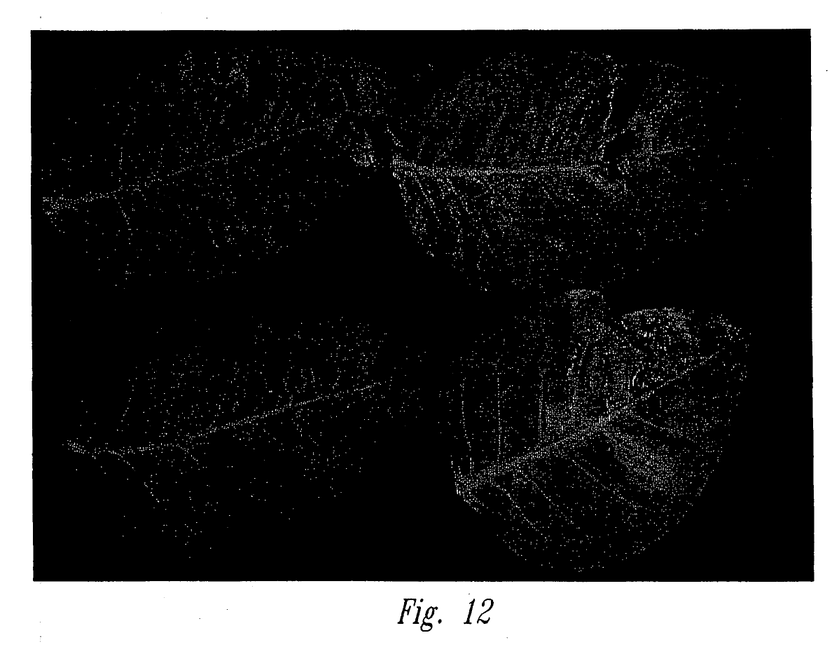

Many plant host-virus combinations also give rise to necrotic local lesions but are not restricted to particular host genotypes. In such combinations the plant is called a local lesion host of the virus. Tobacco is a local lesion host for a number of viruses, including tobacco necrosis virus (TNV) and tomato spotted wilt virus (TSWV). Unlike the TMV and the N gene, the genetic bases of such generic necrotic responses is unknown but it is likely to differ from one virus to another because the morphology of local lesions varies widely. It was of interest, therefore, to also determine the effects of the transgenes on the host response to these disparate plant viruses. Surprisingly, leaves of tobacco lines expressing Bcl-2 or ced-9 had no visible symptoms after inoculation with either TNV or TSWV (FIG. 12). Necrosis was greatly limited on plants expressing IAP after inoculation with each of these viruses as well. Thus, tobacco with either transgene is no longer a local lesion host for these two viruses. Not unexpectedly the same phenotype was noted in both the NN and nn genetic background because the N gene is TMV-specific.

In addition, tobacco etch virus (TEV) has also been tested as described for other viruses above, and demonstrates similar N gene independence as TSWV. Accordingly, Bcl-2 and Ced-9 expressing cells had no visible symptoms of inoculation with TEV (data not shown).

Selected tobacco plants were selfed (i.e., crossed with an same genotype). Importantly, among the progeny plants, those without kanamycin resistance and lacking expression of the transgene were all now susceptible.

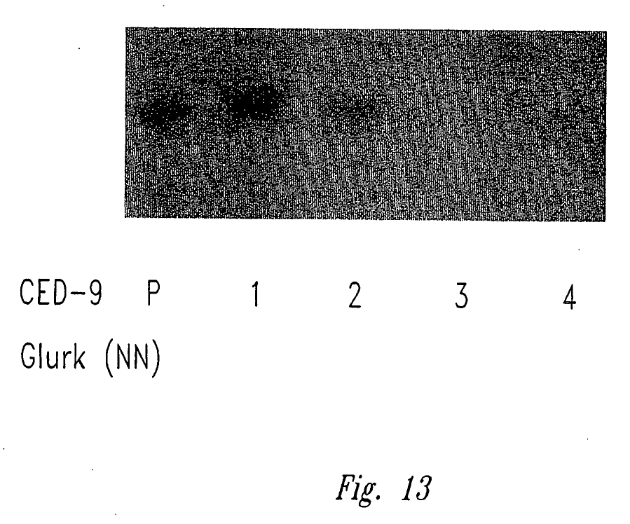

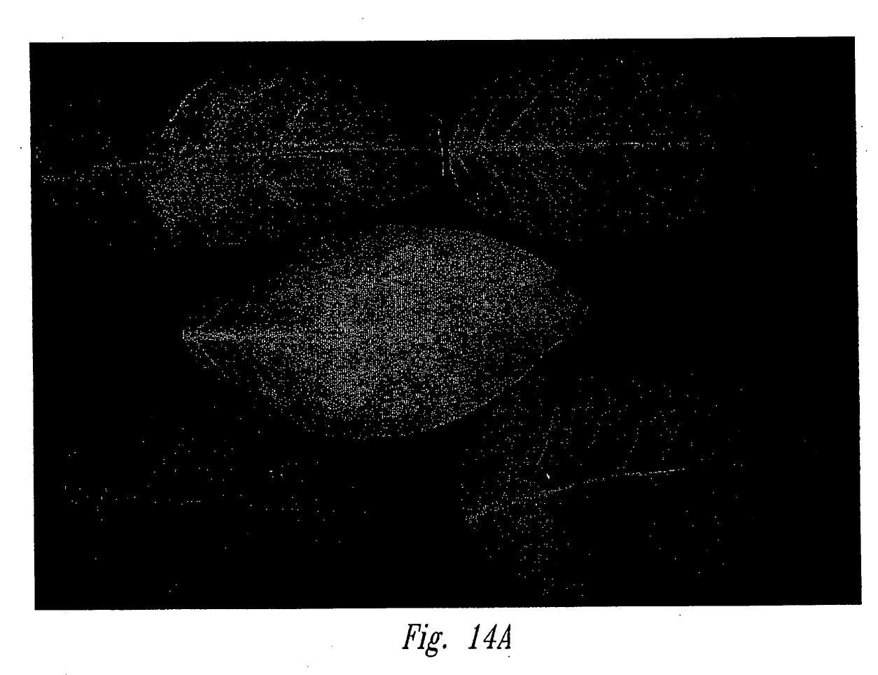

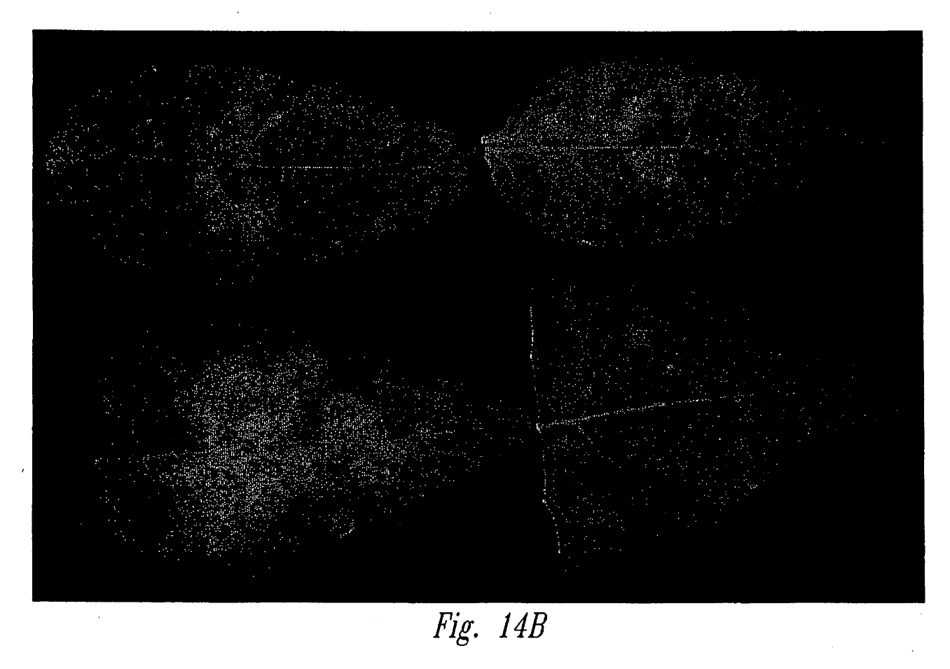

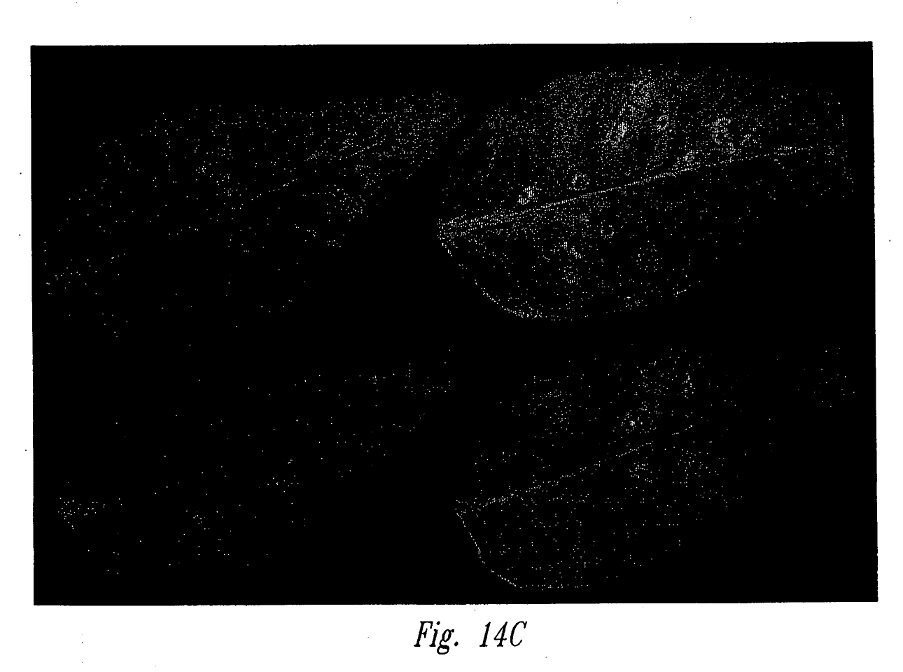

Kanamycin resistant progeny expressing the transgene (see FIGS. 13 (Ced-9 exemplary Bcl-2 and IAP similar), 14A (Bcl-2 expression and TMV inoculation), 14B (Ced-9 expression and TMV inoculation), 14C (Ced-9 expression and TSWV inoculation) were virtually all resistant and identical in phenotype following viral challenge as observed in the parents.

Fungal Assays:

For detached leaf assays, a minimum of 2 leaves per plant from 10 individual plants/transgene of both cultivars were inoculated by placing 5 mm agar plug containing actively growing hyphal tips from 3 day old colonies of either S. sclerotiorum or B. cinerea grown on potato dextrose agar.

Leaves were placed on moistened sterile filter paper in glass petri dishes and incubated for 3-7 days at 25° C. and high humidity. For whole plant inoculations (S. Sclerotiorum only) ascospores (≈10⁴/ml) were incubated for 2 hours in YPSS liquid medium (Tuite, 1968) and sprayed onto tobacco leaves until runoff. Inoculated plants were placed in a mist chamber at 100% relative humidity at 25° C. in the dark. A minimum of five plants/line were used. All experiments were repeated at least three times.

Assays with Sclerotinia sclerotiorum indicated transgenic tobacco were highly tolerant and in most cases were completely resistant unlike wild type tobacco which is highly susceptible. This fungus requires inoculation with a nutritional source, unlike numerous other fungi which can be delivered in water or buffer alone. As shown in FIG. 15, the fungus when inoculated with a rich nutritional source, grows along the tobacco leaf surface, but no infection or host colonization occurs. The fungus eventually ceases growth presumably due to depletion of the nutritional source and importantly even with extended periods of incubation, the fungus is still unable to colonize and infect plant tissue. In contrast the wild type fungus over the same period of time completely colonizes and macerates leaf tissue (FIG. 15).

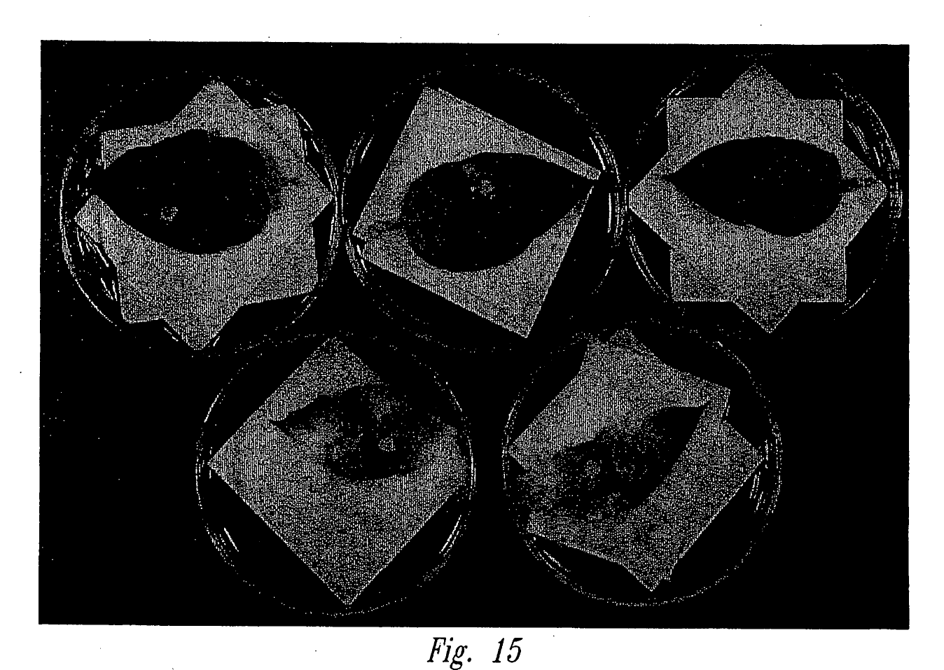

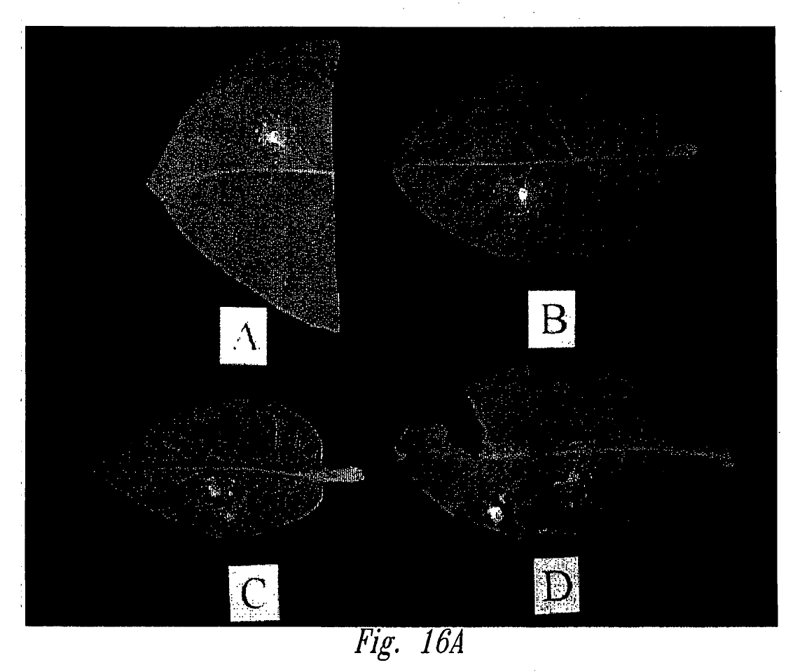

The previously described tobacco plants that were selfed were also evaluated for response to S. sclerotiorum challenge (FIG. 16). Again, progeny plants without kanamycin resistance and lacking transgene expression were all susceptible. Plants selected for kanamycin resistance and transgene expression were generally resistant and identical in phenotype to the parent, primary transformants.

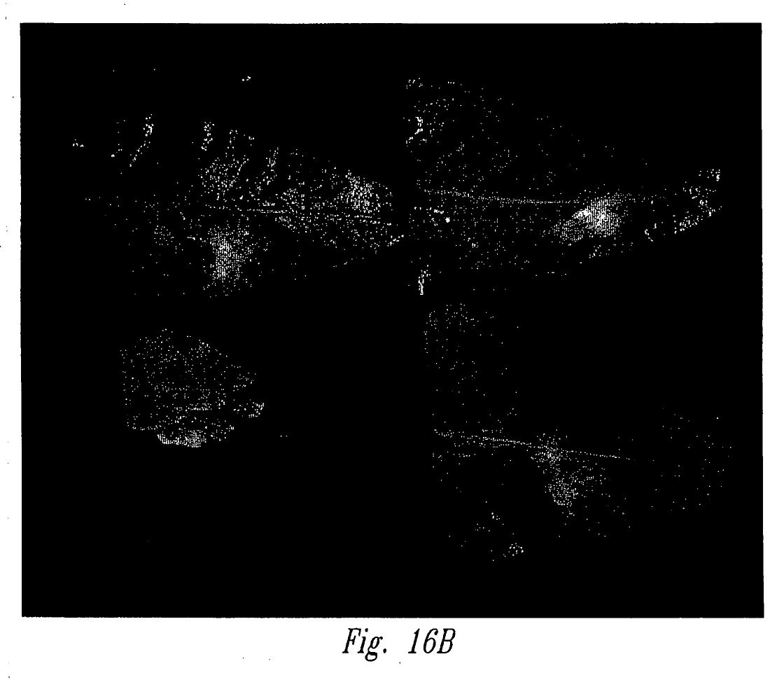

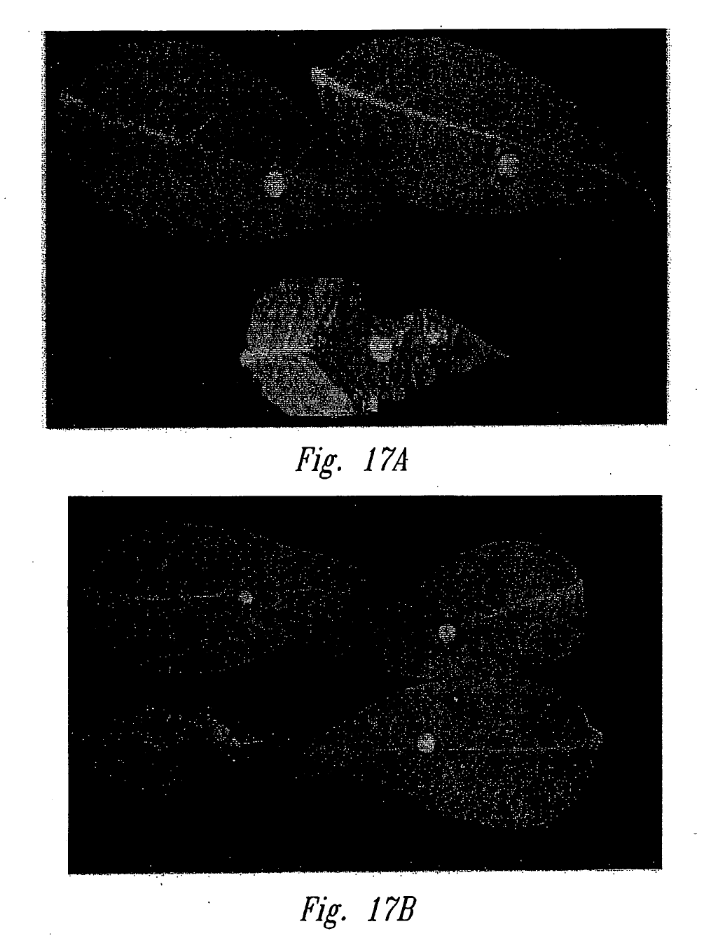

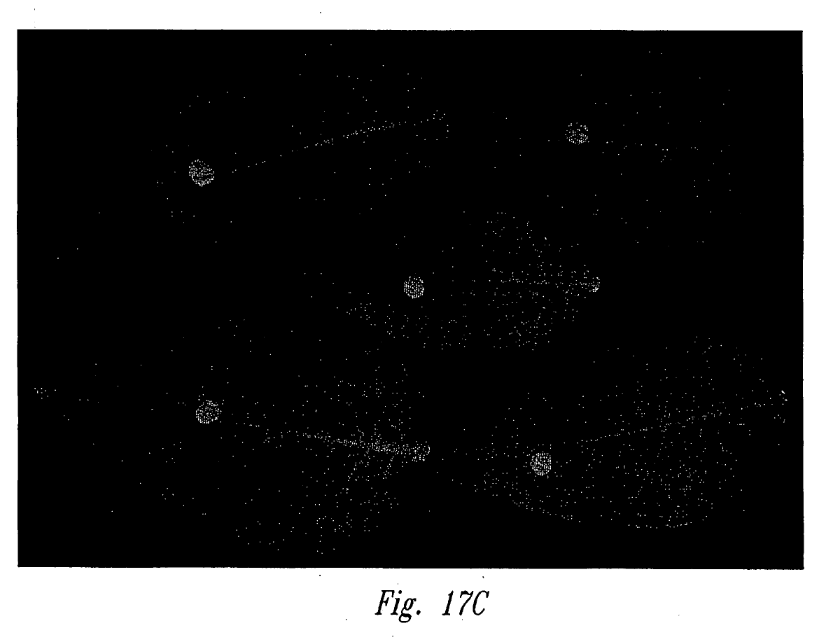

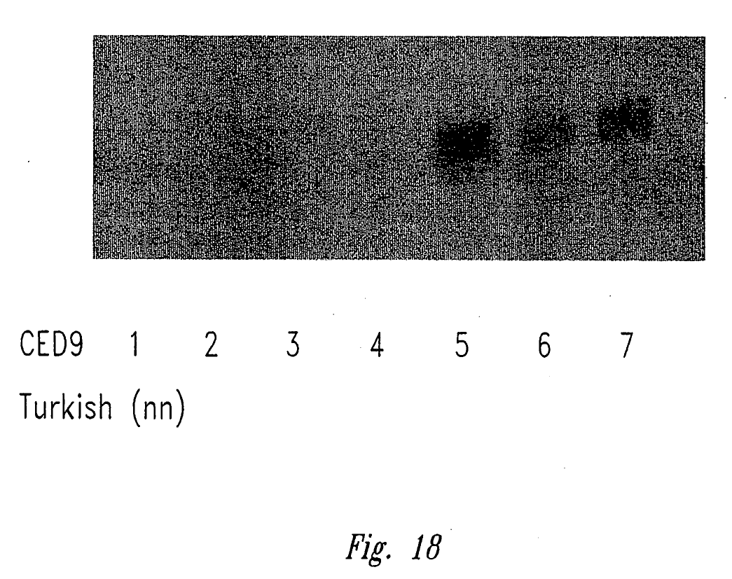

Botrytis cinerea also a broad host range necrotrophic fungal pathogen showed a similar response when inoculated on transgenic tobacco containing any of the three transgenes. In both fungal examples, whole plant inoculations were identical in phenotypic response as to those observed in detached leaf assays (FIGS. 17A-Bcl-2 expressing, 17B-Ced-9 expressing).

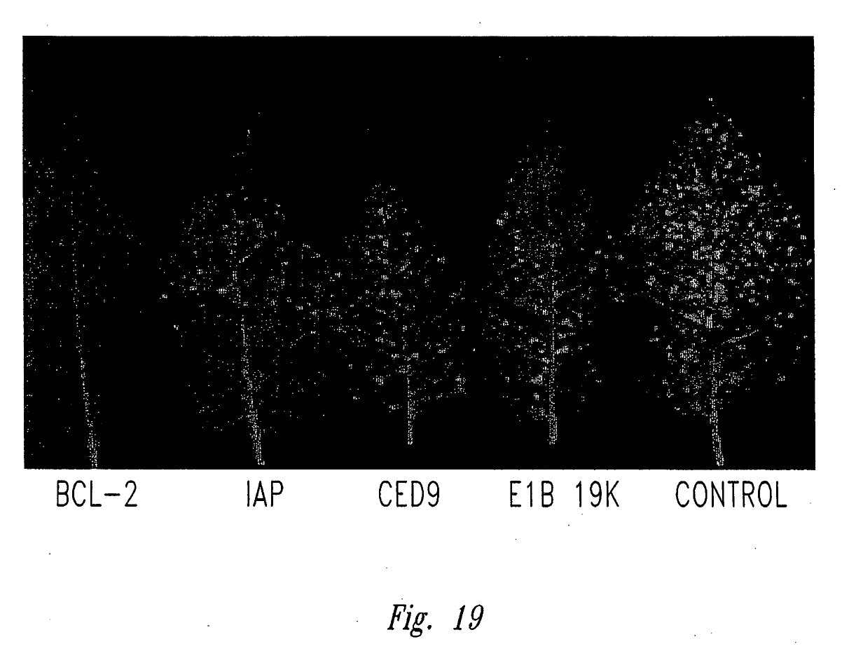

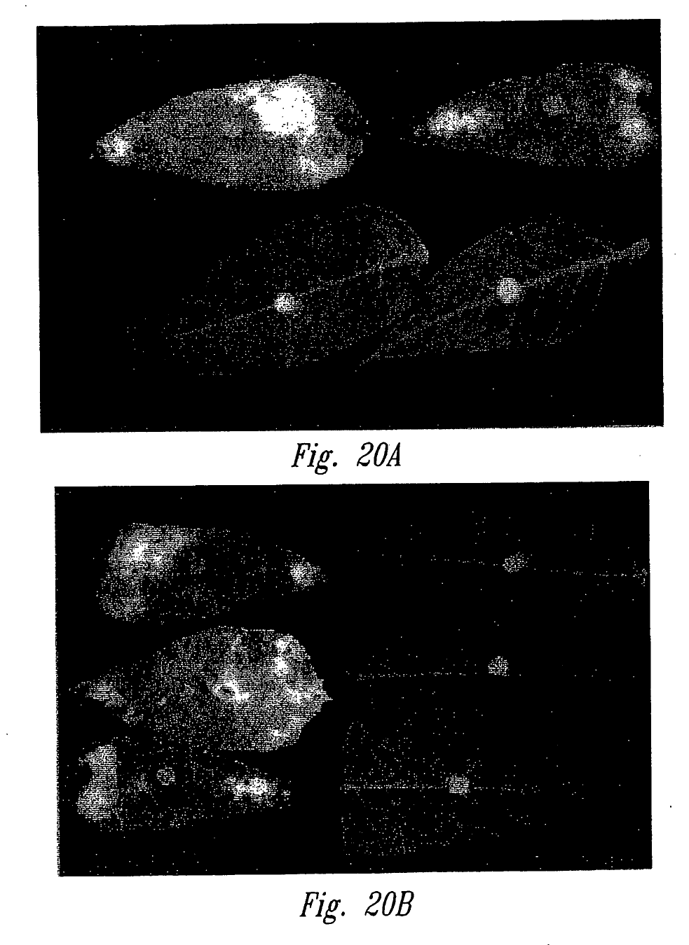

Further, plants encoding the three transgenes Ced-9, Bcl-2, and IAP were resistant to both Cercospora nicotianae (FIG. 19) and Glomerella cingulata in a manner similar to that described for S. sclerotiorum. (data not shown).

Lastly, plants were transformed with a vector encoding wild-type bcl-xL and non-functional mutant form G138A of bcl-xL in the same parent vector as above. Following exposure to S. sclerotiorum.

### Example VIII

### Isolation and Blotting of RNA from Transformed Tobacco Lines

The regenerated kanamycin-resistant strains of transformed tobacco plants were analyzed by hybridizing RNA samples with a radiolabled probe encompassing the apoptotic pathway protein encoding gene of interest, Gordon-Kamm et al., The Plant Cell 2, 603-618, 1990.

Total RNA from the leaves of the transformed tobacco plants was isolated as described, Strommer et al., in: Methods in Plant Molecular Biology and Biotechnology, pp. 49-65, CRC Press, Boca Raton, Fla., 1993. Approximately 600 to 700 mg leaf is cut from a the plant, the midrib removed and the leaf placed on ice. The leaves are ground with liquid nitrogen and transferred to a microcentrifuge tube. 1 ml of TRIzol is added and the leaves are allowed to thaw. The sample is incubated at room temperature for 5 minutes followed by the addition of 0.2 ml of chloroform, mixing, and subsequent incubation for an additional 3 minutes. The samples are then spun at 12,000×g at 4° C. for 15 minutes and the supernatant transferred to a separate tube. To the supernatent 0.5 ml of isopropyl alcohol is added and the resulting solution is gently mixed and incubated for 10 minutes at room temperature. The sample is then spun at 12,000×g at 4° C. for 10 minutes and the supernatant removed. The pellet is washed with 1 ml of 75% ethanol and gently mixed. The washed pellet is spun down at 7,500×g at 4° C. for 5 minutes, the supernatant removed, and the resulting pellet vacuum dried for 3 to 5 minutes or allowed to dry at room temperature for 10 minutes. Subsequently 20 to 50 μl of DEPC H₂O or Formamide is added and incubated at 55° C. for 10 minutes. The samples are then used or kept at −80° C. until needed.

RNA preparations are resolved using gel electrophoresis using standard protocols and transferred to a membrane for hybridization. (see Sambrook et al., supra). First the membrane is prehybridized in a rotary oven at 65° C. for 1 hour with 25 ml of solution comprising (12.5 ml 1M NaHPO₄ pH 7.2, 0.25 g BSA, 50 μl EDTA (0.5 M stock), 1.75 g SDS and ddH₂O to 25 ml (approx. 11.5 ml)). Probes are prepared by klenow labeling with ³²P-dCTP. The probe can then be added directly to the hybridization solution and allowed to incubate overnight at 65° C. in a rotating oven. The membrane is then washed for 10 minutes at room temperature with 50 ml of a low-stringency wash solution (2 ml 1M NaHPO₄, pH 7.2, 0.25 g BSA, 100 μl EDTA (0.5M stock), 2.5 g SDS and ddH2O to 50 ml). The membrane is then washed with one time for 10 minutes at room temperature and two times for 10 minutes at 65° C. with 50ml of a high stringency wash solution (8 ml 1M NaHPO₄, pH 7.2, 400 μl EDTA (0.5M stock), 2 g SDS, and ddH₂O to 200 ml). The membrane is then exposed to X-OMAT AR film for 5 to 10 hours at −80° C. using an intensifying screen.

From the foregoing it will be appreciated that, although specific embodiments of the invention have been described herein for purposes of illustration, various modifications may be made without deviating from the spirit and scope of the invention. Accordingly, the invention is not limited except as by the appended claims.

All references, including journal articles, patents, and patent applications cited within the present application are hereby incorporated herein by reference in their entirety.

1. A method of detecting a nucleic acid molecule that has apoptotic pathway activity in plants, comprising:
a. contacting a population of cells with an inducer of apoptosis, wherein at least some of the cells have been transformed with an expression vector containing a promoter operably associated with a nucleic acid molecule to be expressed, wherein the nucleic acid molecule is not known or suspected to have apoptotic pathway activity;
b. detecting apoptotic pathway activity in the host cells, wherein an increase or decrease in apoptotic pathway activity as compared to a control cell line indicates that the nucleic acid molecule has apoptotic pathway activity; and
c. confirming that the nucleic acid molecule, when expressed in plant cells transformed therewith, exhibits apoptotic pathway activity.

2. A method according to claim 1, wherein the nucleic acid molecule is a DNA molecule.

3. A method according to claim 1, wherein the nucleic acid molecule comprises a cDNA.

4. A method according to claim 1, wherein the population of cells with a plurality of expression vectors, wherein the plurality of expression vectors comprises a plurality of different nucleic acid molecules to be expressed.

5. A method according to claim 1, wherein the inducer of apoptosis is selected from the group consisting of an abiotic stress and a biotic stress.

6. A method according to claim 1, wherein the population of cells contacted with the inducer of apoptosis in part (a) comprise eukaryotic cells.

7. A method according to claim 6, wherein the eukaryotic cells are yeast cells.

8. A method according to claim 1, wherein apoptotic pathway activity is detected in the cells contacted with the inducer of apoptosis by survival of cells transformed with the nucleic acid molecule.

9. A method according to claim 1, wherein the nucleic acid molecule is derived from a source selected from the group consisting of a viral genome, a human genome, a non-human animal genome, a yeast genome, and a plant genome.

10. A method according to claim 1, wherein the confirmation step comprises transforming plant cells with an expression vector containing the nucleic acid molecule operably associated with a promoter and determining whether the transformed plant cells exhibit altered apoptotic activity as compared to control plant cells.

11. An isolated nucleic acid molecule that has apoptotic pathway activity identified using a method according to claim 1.

12. An isolated nucleic acid molecule according to claim 11 that is derived from a source selected from the group consisting of a viral genome, an animal genome, a yeast genome, and a plant genome.

13. An expression vector comprising a promoter operably associated with an isolated nucleic acid molecule according to claim 11.

14. An expression vector according to claim 13, wherein the promoter is selected from the group consisting of a constitutive promoter, an inducible promoter, and a tissue-specific promoter.

15. A transgenic plant cell, wherein the plant cell includes an isolated nucleic acid molecule according to claim 11.

16. A transgenic plant, comprising at least one transgenic plant cell according to claim 15.

17. A transgenic plant cell according to claim 15, wherein the isolated nucleic acid molecule is included within an expression cassette that also comprises a promoter selected from the group consisting of a constitutive promoter, an inducible promoter, and a tissue-specific promoter.

18. A transgenic plant, comprising at least one transgenic plant cell according to claim 17.

19. A plant cultivation method, comprising cultivating a transgenic plant according to claim 16 under conditions suitable for growth of the transgenic plant.

20. A plant cultivation method, comprising cultivating a transgenic plant according to claim 18 under conditions suitable for growth of the transgenic plant.
